SEQUENCE LISTING (28 sequences)

SEQ ID NO: 1 - DNA, 2734 bp, Artificial Sequence
  Synthetic
datggtgcttctgtggtgtgtagtgagtctctacttttatggaatcctgcaaagtgatgcctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtgtccggtggcgcgcctatgctgagcgaggctgataaatgcaaggaacgtgaagaaaaaataattttagtgtcatctgcaaatgaaattgatgttcgtccctgtcctcttaacccaaatgaacacaaaggcactataacttggtataaggatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgagagtagattttataaacatccatttacctgttttgccaagaatacacatggtatagatgcagcatatatccagttaatatatccagtcactaattccggagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - protein, 911 aa, Artificial Sequence
  Synthetic
PMVLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVSGGAPMLSEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 2704 bp, Artificial Sequence
  Synthetic
datggtgttactcagacttatttgtttcatagctctactgatttcttctctggaggctgataaatgcaaggaacgtgaagaaaaaataattttagtgtcatctgcaaatgaaattgatgttcgtccctgtcctcttaacccaaatgaacacaaaggcactataacttggtataaggatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgagagtagattttataaacatccatttacctgttttgccaagaatacacatggtatagatgcagcatatatccagttaatatatccagtcactaattcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtggaatccggagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 4 - protein, 901 aa, Artificial Sequence
  Synthetic
PMVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNSERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVESGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 2710 bp, Artificial Sequence
  Synthetic
datggtgttactcagacttatttgtttcatagctctactgatttcttctctggaggctgataaatgcaaggaacgtgaagaaaaaataattttagtgtcatctgcaaatgaaattgatgttcgtccctgtcctcttaacccaaatgaacacaaaggcactataacttggtataaggatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgagagtagattttataaacatccatttacctgttttgccaagaatacacatggtatagatgcagcatatatccagttaatatatccagtcactaattcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtggaatccggagagtccaaatacggtccgccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 6 - protein, 903 aa, Artificial Sequence
  Synthetic
PMVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNSERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVESGESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - DNA, 2710 bp, Artificial Sequence
  Synthetic
datggtgttactcagacttatttgtttcatagctctactgatttcttctctggaggctgataaatgcaaggaacgtgaagaaaaaataattttagtgtcatctgcaaatgaaattgatgttcgtccctgtcctcttaacccaaatgaacacaaaggcactataacttggtataaggatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgagagtagattttataaacatccatttacctgttttgccaagaatacacatggtatagatgcagcatatatccagttaatatatccagtcactaattcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtggaatccggagagtccaaatacggtccgccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 8 - protein, 903 aa, Artificial Sequence
  Synthetic
PMVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNSERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVESGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - DNA, 2704 bp, Artificial Sequence
  Synthetic
datggtgcttctgtggtgtgtagtgagtctctacttttatggaatcctgcaaagtgatgcctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtggaaaaatgcaaggaacgtgaagaaaaaataattttagtgagctcagcaaatgaaatcgatgttcgtccctgtcctcttaacccaaatgaacacaaaggcactataacttggtataaggatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgagagtagattttataaacatccatttacctgttttgccaagaatacacatggtatagatgcagcatatatccagttaatatatccagtcactaattccggagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 10 - protein, 901 aa, Artificial Sequence
  Synthetic
PMVLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVEKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 2710 bp, Artificial Sequence
  Synthetic
datggtgcttctgtggtgtgtagtgagtctctacttttatggaatcctgcaaagtgatgcctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtggaaaaatgcaaggaacgtgaagaaaaaataattttagtgagctcagcaaatgaaatcgatgttcgtccctgtcctcttaacccaaatgaacacaaaggcactataacttggtataaggatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgagagtagattttataaacatccatttacctgttttgccaagaatacacatggtatagatgcagcatatatccagttaatatatccagtcactaattccggagagtccaaatacggtccgccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 12 - protein, 903 aa, Artificial Sequence
  Synthetic
PMVLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVEKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNSGESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - DNA, 2710 bp, artificial
  synthetic
datggtgcttctgtggtgtgtagtgagtctctacttttatggaatcctgcaaagtgatgcctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtggaaaaatgcaaggaacgtgaagaaaaaataattttagtgagctcagcaaatgaaatcgatgttcgtccctgtcctcttaacccaaatgaacacaaaggcactataacttggtataaggatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgagagtagattttataaacatccatttacctgttttgccaagaatacacatggtatagatgcagcatatatccagttaatatatccagtcactaattccggagagtccaaatacggtccgccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 14 - protein, 903 aa, Artificial Sequence
  Synthetic
PMVLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVEKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNSGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - DNA, 2749 bp, Artificial Sequence
  Synthetic
datggtgcgcttgtacgtgttggtaatgggagtttctgccttcacccttcagcctgcggcacacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccatgtaaggtgtttctgggaaccggcacacccttaaccaccatgctgtggtggacggccaatgacacccacatagagagcgcctacccgggaggccgcgtgaccgaggggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtgtccggagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 16 - protein, 916 aa, Artificial Sequence
  Synthetic
PMVRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 2755 bp, Artificial Sequence
  Synthetic
datggtgcgcttgtacgtgttggtaatgggagtttctgccttcacccttcagcctgcggcacacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccatgtaaggtgtttctgggaaccggcacacccttaaccaccatgctgtggtggacggccaatgacacccacatagagagcgcctacccgggaggccgcgtgaccgaggggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtgtccggagagtccaaatacggtccgccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 18 - protein, 918 aa, Artificial Sequence
  Synthetic
PMVRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVSGESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 2755 bp, Artificial Sequence
  Synthetic
datggtgcgcttgtacgtgttggtaatgggagtttctgccttcacccttcagcctgcggcacacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccatgtaaggtgtttctgggaaccggcacacccttaaccaccatgctgtggtggacggccaatgacacccacatagagagcgcctacccgggaggccgcgtgaccgaggggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtgtccggagagtccaaatacggtccgccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 20 - protein, 918 aa, Artificial Sequence
  Synthetic
PMVRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVSGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - DNA, 2749 bp, Artificial Sequence
  Synthetic
datggtgcttctgtggtgtgtagtgagtctctacttttatggaatcctgcaaagtgatgcctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtgcacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccatgtaaggtgtttctgggaaccggcacacccttaaccaccatgctgtggtggacggccaatgacacccacatagagagcgcctacccgggaggccgcgtgaccgaggggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctccggagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 22 - protein, 916 aa, Artificial Sequence
  Synthetic
PMVLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 2755 bp, Artificial Sequence
  Synthetic
datggtgcttctgtggtgtgtagtgagtctctacttttatggaatcctgcaaagtgatgcctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtgcacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccatgtaaggtgtttctgggaaccggcacacccttaaccaccatgctgtggtggacggccaatgacacccacatagagagcgcctacccgggaggccgcgtgaccgaggggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctccggagagtccaaatacggtccgccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 24 - protein, 918 aa, Artificial Sequence
  Synthetic
PMVLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSGESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - DNA, 2755 bp, Artificial Sequence
  Synthetic
datggtgcttctgtggtgtgtagtgagtctctacttttatggaatcctgcaaagtgatgcctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtgcacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccatgtaaggtgtttctgggaaccggcacacccttaaccaccatgctgtggtggacggccaatgacacccacatagagagcgcctacccgggaggccgcgtgaccgaggggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctccggagagtccaaatacggtccgccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 26 - protein, 918 aa, Artificial Sequence
  Synthetic
PMVLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - DNA, 2734 bp, Artificial Sequence
  Synthetic
datggtgcttctgtggtatttgatgagtctgtccttctatgggatcctgcagagtcatgcttcggagcgctgtgatgactggggactagataccatgcgacaaatccaagtgtttgaagatgagccggctcgaatcaagtgccccctctttgaacacttcctgaagtacaactacagcactgcccattcctctggccttaccctgatctggtactggaccaggcaagaccgggacctggaggagcccattaacttccgcctcccagagaatcgcatcagtaaggagaaagatgtgctctggttccggcccaccctcctcaatgacacgggcaattacacctgcatgttgaggaacacaacttactgcagcaaagttgcatttcccctggaagttgttcagaaggacagctgtttcaattctgccatgagattcccagtgcacaagatgtatattgaacatggcattcataagatcacatgtccaaatgtagacggatactttccttccagtgtcaaaccatcggtcacttggtataagggttgtactgaaatagtggactttcataatgtactacccgagggcatgaacttgagctttttcatccccttggtttcaaataacggaaattacacatgtgtggttacatatcctgaaaacggacgtctctttcacctcaccaggactgtgactgtaaaggtggtgggctcaccaaaggatgcattgccaccccagatctattctccaaatgaccgtgttgtctatgagaaagaaccaggagaggaactggttattccctgcaaagtctatttcagtttcattatggactcccacaatgaggtctggtggaccattgatggaaagaagcctgatgacgtcacagtcgacatcactattaatgaaagtgtaagttattcttcaacggaagatgaaacaaggactcagattttgagcatcaagaaagtcaccccggaggatctcaggcgcaactatgtctgtcatgctcgaaataccaaaggggaagctgagcaggctgccaaggtgaaacagaaagtcataccaccaaggtacacagtactggagattgacgtatgtacagaatatccaaatcagatcgttttgtttttatctgtaaatgaaattgatattcgcaagtgtcctcttactccaaataaaatgcacggcgacaccataatttggtacaagaatgacagcaagacccccatatcagcggaccgggactccaggattcatcagcagaatgaacatctttggtttgtacctgccaaggtggaggactcaggatattactattgtatagtaagaaactcaacttactgcctcaaaactaaagtaaccgtaactgtgttagagaatgaccctggcttgtgttacagcacacaggccaccttcccacagcggctccacattgccggggatggaagtcttgtgtgcccttatgtgagttattttaaagatgaaaataatgagttacccgaggtccagtggtataagaactgtaaacctctgcttcttgacaacgtgagcttcttcggagtaaaagataaactgttggtgaggaatgtggctgaagagcacagaggggactatatatgccgtatgtcctatacgttccgggggaagcaatatccggtcacacgagtaatacaatttatcacaatagatgaaaacaagagggacagacctgttatcctgagccctcggaatgagacgatcgaagctgacccaggatcaatgatacaactgatctgcaacgtcacgggccagttctcagaccttgtctactggaagtggaatggatcagaaattgaatggaatgatccatttctagctgaagactatcaatttgtggaacatccttcaaccaaaagaaaatacacactcattacaacacttaacatttcagaagttaaaagccagttttatcgctatccgtttatctgtgttgttaagaacacaaatatttttgagtcggcgcatgtgcagttaatatacccagtccctggcccgggcgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 28 - protein, 911 aa, Artificial Sequence
  Synthetic
PMVLLWYLMSLSFYGILQSHASERCDDWGLDTMRQIQVFEDEPARIKCPLFEHFLKYNYSTAHSSGLTLIWYWTRQDRDLEEPINFRLPENRISKEKDVLWFRPTLLNDTGNYTCMLRNTTYCSKVAFPLEVVQKDSCFNSAMRFPVHKMYIEHGIHKITCPNVDGYFPSSVKPSVTWYKGCTEIVDFHNVLPEGMNLSFFIPLVSNNGNYTCVVTYPENGRLFHLTRTVTVKVVGSPKDALPPQIYSPNDRVVYEKEPGEELVIPCKVYFSFIMDSHNEVWWTIDGKKPDDVTVDITINESVSYSSTEDETRTQILSIKKVTPEDLRRNYVCHARNTKGEAEQAAKVKQKVIPPRYTVLEIDVCTEYPNQIVLFLSVNEIDIRKCPLTPNKMHGDTIIWYKNDSKTPISADRDSRIHQQNEHLWFVPAKVEDSGYYYCIVRNSTYCLKTKVTVTVLENDPGLCYSTQATFPQRLHIAGDGSLVCPYVSYFKDENNELPEVQWYKNCKPLLLDNVSFFGVKDKLLVRNVAEEHRGDYICRMSYTFRGKQYPVTRVIQFITIDENKRDRPVILSPRNETIEADPGSMIQLICNVTGQFSDLVYWKWNGSEIEWNDPFLAEDYQFVEHPSTKRKYTLITTLNISEVKSQFYRYPFICVVKNTNIFESAHVQLIYPVPGPGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

(19) United States
(12) Patent Application Publication
(10) Pub. No.: US 2016/0120941 A1
(43) Pub. Date: May 5, 2016

(54) METHODS OF USING IL-1 ANTAGONISTS TO TREAT ALZHEIMER'S DISEASE

(71) Applicant: REGENERON PHARMACEUTICALS, INC., Tarrytown, NY (US)

(72) Inventors: Susan D. Croll, Putnam Valley, NY (US); Stanley Wiegand, Hopewell Junction, NY (US)

(73) Assignee: REGENERON PHARMACEUTICALS, INC., Tarrytown, NY (US)

(21) Appl. No.: 14/893,932

(22) PCT Filed: May 30, 2014
(86) PCT No.: PCT/US2014/040160

§ 371 Date: Nov. 24, 2015

Related U.S. Application Data
(60) Provisional application No. 61/829,713, filed on May 31, 2013.

(51) Int. Cl. A61K 38/17; A61K 31/427; A61K 45/06; C07K 14/715; A61K 39/395

(52) U.S. Cl. CPC A61K 38/1793; C07K 14/7155; A61K 39/3955; A61K 45/06; A61K 31/427; C07K 2319/30

(57) ABSTRACT

The invention provides methods of treating, inhibiting, or ameliorating a disease characterized by aberrant deposition of beta amyloid in a subject in need thereof, comprising administering to a subject a therapeutic amount of an interleukin 1 (IL-1) antagonist, wherein the disease, or condition is treated, inhibited, or ameliorated, or wherein the onset or progression of the disease, or at least one symptom of the disease, is delayed. The IL-1 antagonist is an IL-1 trap, preferably comprising a sequence selected from the group consisting of SEQ ID NO: 2, 4, 6, 8, 10, 12, 14, 16, 18, 20, 22, 24, 26, and 28 and capable of binding and inhibiting IL-1. The therapeutic methods are useful for treating a human adult suffering from Alzheimer's Disease or cerebral amyloid angiopathy.

### FIELD OF THE INVENTION

The invention relates to methods of using an interleukin-1 (IL-1) antagonist to treat or to slow the progression of a disease characterized in part by beta amyloid (Aβ) expression or activity, or by aberrant deposition of beta amyloid in a subject, such as in Alzheimer's disease, and more specifically, the pathologies associated with such a disease, including for example, behavioral changes or cognitive dysfunction associated with Alzheimer's disease.

### STATEMENT OF RELATED ART

The proinflammatory cytokine interleukin-1 (IL-1) is an important player in inflammatory processes throughout the body, including in the central nervous system (CNS). IL-1 is found in two distinct isoforms, IL-1a and IL-β, although IL-β is considered the primary active isoform. Upregulation of IL-β is part of the response to a range of CNS insults, including infections, stroke, and traumatic injuries (Allan, S. M., Tyrrell, P. J., & Rothwell, N. J. (2005), Nature Reviews Immunology, 5, 629-640). This neuroinflammatory response is characterized by activation of resident glial cells (microglia and astrocytes), infiltration of peripheral immune cells, and the expression of inflammatory mediators, such as cytokines and chemokines (Shaftel, S. S., Griffin, W. S. T., & O'Banion, M. K. (2008), Journal of Neuroinflammation, 5:7).

IL-1-mediated neuroinflammation may also play a role in the pathogenesis of neurodegenerative diseases. For example, elevated levels of IL-1 are reported in the brain tissue of patients with Alzheimer's disease (AD). AD (Griffin, W. S., Stanley, L. C., Ling, C., White, L., MacLeod, V., Perrot, L. J., Araoz, C. (1989), Proceedings of the National Academy of Sciences of the United States of America, 86, 7611-7615) and in rodent models of the disease (Benzing, W. C., Wujek, J. R., Ward, E. K., Shaffer, D., Ashe, K. H., Younkin, S. G., & Brunden, K. R. (1999), Neurobiology of Aging, 20(6), 581-589). The degree of neuroinflammation and IL-1 expression has been shown to correlate with the level of pathology in AD patients (Sheng, J. G., Ito, K., Skinner, R. D., Mrak, R. E., Rovnaghi, C. R., Van Eldik, L. J., & Griffin, W. S. (1996), Neurobiology of Aging, 17(5), 761-766).

Although the research to date may support a link between AD and IL-1, it is unclear what role IL-1 may play. IL-1 modulates actions that contribute to AD pathology, including the synthesis and processing of the Aβ precursor protein and the activity of acetylcholinesterase (Mrak, R. E. & Griffin, W. S. (2001), Neurobiology of Aging, 22(6), 903-908). Chronic IL-1 expression has been associated with demyelination (Ferrari, C. C., Depino, A. M., Prada, F., Muraro, N., Camptbell, S., Podhajcer, O., Pitossi, F. J. (2004), American Journal of Pathology, 165(5), 1827-1837), breakdown of the blood-brain barrier, and neutrophil recruitment (Ferrari et al, supra; Shaftel, S. S., Carlson, T. J., Olschowka, J. A., Kyrkanides, S., Matousek, S. B., & O'Banion, M. K. (2007), Journal of Neuroscience, 27(35), 9301-9309). IL-1 also activates microglia, which in turn produce pro-inflammatory cytokines such as IL-1, IL-6, and tumor necrosis factor alpha (TNFα). Neuronal insults, such as the accumulation of Aβ, may therefore induce a self-propagating cycle of cytokine activation in which levels of IL-1 constantly rise, leading to neuronal damage and further plaque deposition (Griffin, W. S. T., Sheng, J. G., Royston, M. C., Gentelman, S. M., McKenzie, J. E., Graham, D. I., Mrak, R. E. (1998), Brain Pathology, 8, 65-72).

Prior research has explored the use of anti-inflammatory drugs as a therapeutic strategy against AD. Epidemiological studies have demonstrated a reduced risk of developing the disease in long-term non-steroidal anti-inflammatory drug (NSAID) users (Szekely, C. A., Breitner, J. C. S., Fitzpatrick, A. L., Rea, T. D., Psaty, B. M., Kuller, L. H., & Zandi, P. P. (2008), Neurology, 70(1), 17-24). In transgenic animal models, chronic NSAID administration has been somewhat effective in preventing or delaying the onset of amyloid deposition, dystrophic neurite formation and inflammation (Lim, G. P., Yang, F., Chu, T., Chen, P., Beech, W., Teter, B., Cole, G. M. (2000), Journal of Neuroscience, 20(15), 5709-5714). However, randomized clinical trials have failed to consistently support the therapeutic effectiveness of NSAIDs against AD (Scharf, S., Mander, A., Ugoni, A., Vajda, F., & Christophidis, N. (1999), Neurology, 53(1), 197-201; Aisen, P. S., Schafer, K. A., Grundman, M., Pfeiffer, E., Sano, M., Davis, K. L., Thal, L. J. (2003), Journal of the American Medical Association, 289(21), 2819-2826).

Given the small number of approved therapies to treat, or to slow down the progression of a disease characterized in part by beta amyloid expression, activity, or aberrant deposition, such as AD, there is a need to identify and explore the use of other agents for treating these diseases, such as the IL-1 antagonists as described herein.

### BRIEF SUMMARY OF THE INVENTION

The invention provides a method for treating a subject suffering from a disease characterized in part by the deposition and/or activity of beta amyloid in the brain tissue of a subject by administering an interleukin-1 (IL-1) antagonist. An IL-1 antagonist is a compound capable of blocking or inhibiting at least one biological activity of IL-1. An IL-1 antagonist may take the form of an antibody, a soluble receptor, or a fusion protein capable of trapping IL-1, such as an IL-1 trap as described herein. In one embodiment, the subject is a human patient suffering from Alzheimer's Disease (AD). The IL-1 trap may be administered alone, or in conjunction with one or more therapeutic agents that are useful for treating AD, or for slowing the progression of the disease, or for ameliorating at least one symptom associated with the disease, including, but not limited to behavioral changes associated with AD, or the cognitive decline or dysfunction observed in patients with AD.

Accordingly, in a first aspect, the invention features a method for treating, or delaying the onset, or the progression of a disease characterized in part by beta amyloid expression, activity, or deposition in a subject in need thereof, or for ameliorating at least one symptom associated with the disease, the method comprising administering to the subject a therapeutically effective amount of an IL-1 antagonist as a first therapeutic agent, wherein the IL-1 antagonist is selected from the group consisting of an antibody specific for IL-1 alpha or IL-1 beta, or an antigen-binding fragment thereof, a soluble IL-1 receptor, and an IL-1 trap, wherein the IL-1 trap is a fusion protein comprising an IL-1 binding portion of the extracellular domain of IL-1 RAcP, an IL-1 binding portion of the extracellular domain of IL-1 R1, and a multimerizing component.

In one embodiment, the invention provides a method for treating, or delaying the onset, or the progression of a disease characterized in part by beta amyloid expression, activity, or deposition in a subject in need thereof, or for ameliorating at least one symptom associated with the disease, the method comprising administering to the subject a therapeutically effective amount of an IL-1 antagonist as a first therapeutic agent, wherein the IL-1 antagonist is an IL-1 trap, wherein the IL-1 trap is a fusion protein comprising an IL-1 binding portion of the extracellular domain of IL-1 RAcP, an IL-1 binding portion of the extracellular domain of IL-1 R1, and a multimerizing component.

In a related aspect, the invention features a method of inhibiting IL-1 activity for treating a disease, or delaying the onset or the progression of a disease characterized in part by beta amyloid expression, activity, or deposition in a subject in need thereof, or for ameliorating at least one symptom associated with the disease, the method comprising administering to the subject a therapeutically effective amount of an IL-1 antagonist as a first therapeutic agent.

In one embodiment, the first therapeutic agent is an IL-1 antagonist selected from the group consisting of an antibody specific for IL-1 alpha or IL-1 beta, a soluble IL-1 receptor that blocks or inhibits the activity of IL-1 alpha and/or beta, or an IL-1 fusion protein (e.g. an IL-1 trap as described herein).

In one particular embodiment, the IL-1 antagonist is a fusion protein comprising an IL-1 binding portion of the extracellular domain of IL-1 Receptor Accessory protein (IL-1RAcP), an IL-1 binding portion of the extracellular domain of IL-1 R1, and a multimerizing component.

In one embodiment, the IL-1 antagonist is an IL-1-specific fusion protein comprising two IL-1 receptor components and a multimerizing component, for example, an IL-1 trap as described in U.S. Pat. Nos. 6,927,044; 6,472,179; 7,459,426; 8,414,876; 7,361,350; 8,114,394; 7,820,154 and 7,632,490, all of which are specifically incorporated by reference in their entirety.

In one embodiment, the IL-1 trap is the fusion protein shown in SEQ ID NO: 2, 4, 6, 8, 10, 12,14, 16, 18, 20, 22, 24, 26 and 28. In one embodiment, the IL-1 trap is shown in SEQ ID NO: 28. In one embodiment, the IL-1 trap is shown in SEQ ID NO: 10. The invention encompasses the use of an IL-1 trap substantially identical to the protein of SEQ ID NO: 2, 4, 6, 8, 10, 12,14, 16, 18, 20, 22, 24, 26, 28, that is, a protein having at least 95% identity, at least 97% identity, at least 98% identity to the protein of SEQ ID NO: 2, 4, 6, 8, 10, 12, 14, 16, 18, 20, 22, 24, 26, and 28 and capable of binding and inhibiting IL-1. Further, in specific embodiments, the IL-1 antagonist is a modified IL-1 trap comprising one or more receptor components and one or more immunoglobulin-derived components specific for IL-1 and/or an IL-1 receptor. In another embodiment, the IL-1 antagonist is a modified IL-1 trap comprising one or more immunoglobulin-derived components specific for IL-1 and/or an IL-1 receptor.

The subject being treated is most preferably a human suffering from a disease associated with beta-amyloid deposition and/or activity in the brain, such as Alzheimer's disease. Other subjects that may benefit from such therapy include subjects suffering from multi-infarct dementia, cognitive impairment, Down's syndrome and cerebral amyloid angiopathy. In certain embodiments the Alzheimer's disease may be prodromal, preclinical or clinical stage AD. In certain embodiments the cerebral amyloid angiopathy may be preclinical or clinical stage cerebral amyloid angiopathy.

In one embodiment, the IL-1 trap is a fusion protein comprising the amino acid sequence of SEQ ID NO: 10.

In one embodiment, the IL-1 trap is a fusion protein comprising the amino acid sequence of SEQ ID NO: 28.

In certain embodiments, the administration of the IL-1 trap is subcutaneous, intramuscular, intranasal, intraarterial, intravenous, intrathecal, intraventricular, intracerebral, topical, transdermal administration or oral.

In one embodiment, a therapeutically effective amount of the IL-1 trap to be administered is between about 1 mg/kg to about 750 mg/kg.

In one embodiment, a therapeutically effective amount of the IL-1 trap to be administered is between about 10 mg/kg to about 500 mg/kg.

In one embodiment, a therapeutically effective amount of the IL-1 trap to be administered is between about 50 mg/kg to about 150 mg/kg.

In certain embodiments the methods of the invention provide for treating, inhibiting, or ameliorating a disease, or delaying the onset, or the progression of a disease characterized in part by beta amyloid expression, activity, or deposition in a subject in need thereof by administering a therapeutically effective amount of an IL-1 antagonist/trap, as described herein, as a first therapeutic agent and a therapeutically effective amount of one or more other therapeutic agents, wherein the disease or at least one symptom associated with the disease is lessened in severity or duration, or wherein the onset or progression of the disease or at least one symptom associated with the disease is delayed.

In certain embodiments the at least one symptom associated with the disease is selected from the group consisting of memory loss, depression, anxiety, dementia, irritability, confusion, inattention, mood swings, and aggressive and/or apathetic behavior.

In certain embodiments the other therapeutic agent(s) is/are administered by any route selected from subcutaneous, intramuscular, intranasal, intraarterial, intravenous, intrathecal, intraventricular, intracerebral, topical, transdermal administration or oral.

In one embodiment, the other therapeutic agent is an acetylcholinesterase inhibitor or a glutamate pathway modifier.

In one embodiment, the acetylcholinesterase inhibitor is selected from the group consisting of ARICEPT® (donepezil HCl), EXELON® (rivastigmine tartrate), and RAZADYNE® (galantamine HBr).

In one embodiment, the glutamate pathway modifier is Namenda (memantine).

In one embodiment, the other therapeutic agent(s) is/are selected from the group consisting of a different IL-1 antagonist, an anti-inflammatory agent, an antibody specific for tau, an antibody specific for beta amyloid and a microtubule stabilizer.

In one embodiment, the other therapeutic agent(s) is/are a different IL-1 antagonist selected from the group consisting of an IL-1 alpha or IL-1 beta antibody, a soluble IL-1 receptor, a different IL-1 trap, anakinra (KINERET®) and canakinumab.

In one embodiment, the anti-inflammatory agent is aspirin or a different NSAID.

In one embodiment, the antibody specific for beta amyloid is selected from the group consisting of solanezumab, gantenerumab, and bapineuzumab.

In one embodiment, the microtubule stabilizer is epothilone.

A second aspect provides a method of improving cognitive impairment in a mammal having beta amyloid deposits in brain tissue, the method comprising administering to the subject a therapeutically effective amount of an IL-1 antagonist as a first therapeutic agent, wherein the IL-1 antagonist is selected from the group consisting of an antibody specific for IL-1 alpha or IL-1 beta, or an antigen binding fragment thereof, a soluble IL-1 receptor, and an IL-1 fusion protein (IL-1 trap).

In a related aspect, the invention provides a method of improving cognitive impairment in a mammal having beta amyloid deposits in brain tissue, the method comprising administering to the subject a therapeutically effective amount of an IL-1 antagonist as a first therapeutic agent, wherein the IL-1 antagonist is an IL-1 fusion protein (IL-1 trap).

In one embodiment, the IL-1 antagonist is a fusion protein comprising an IL-1 binding portion of the extracellular domain of IL-1 RAcP, an IL-1 binding portion of the extracellular domain of IL-1 R1, and a multimerizing component, wherein the mammal demonstrates an improvement in cognitive function(s) without the necessity of a change in the beta amyloid plaque burden in the brain.

In one embodiment, the invention provides for a method of improving cognitive impairment in a mammal having beta amyloid deposits in brain tissue, the method comprising administering a composition comprising an IL-1 trap of the invention as a first therapeutic agent, either alone, or in combination with one or more other therapeutic agents useful for treating the disease or at least one symptom of the disease. In one embodiment, the method provides for improvement of cognitive impairment in a subject having beta amyloid deposits in the brain, without necessarily altering the amount (increase or decrease) of beta amyloid in the brain. The improvement of cognitive impairment in a subject may be an improvement in learning performance, or an improvement in memory performance, or a decrease in memory loss, or a decrease in learning impairment.

In one embodiment, the cognitive impairment is associated with Alzheimer's disease. In certain embodiments, the treatment results in slowing the progression of any one or more cognitive or non-cognitive behavioral changes in the subject, including but not limited to memory loss, inability to learn, depression, anxiety, dementia, irritability, confusion, inattention, mood swings, diminished general locomotor and/or exploratory activity and aggressive and/or apathetic behavior. Other subjects that may benefit from therapy with an IL-1 trap of the invention in combination with one or more other therapeutic agents include subjects suffering from multi-infarct dementia, cognitive impairment, Down's syndrome and cerebral amyloid angiopathy. In certain embodiments the Alzheimer's disease may be prodromal, preclinical or clinical stage AD. In certain embodiments the cerebral amyloid angiopathy may be preclinical or clinical stage cerebral amyloid angiopathy.

In one embodiment, the IL-1 trap is the fusion protein shown in SEQ ID NO: 2, 4, 6, 8, 10, 12,14, 16, 18, 20, 22, 24, 26 and 28. In one embodiment, the IL-1 trap is shown in SEQ ID NO: 28. In one embodiment, the IL-1 trap is shown in SEQ ID NO: 10. The invention encompasses the use of an IL-1 trap substantially identical to the protein of SEQ ID NO: 2, 4, 6, 8, 10, 12,14, 16, 18, 20, 22, 24, 26, 28, that is, a protein having at least 95% identity, at least 97% identity, at least 98% identity to the protein of SEQ ID NO: 2, 4, 6, 8, 10, 12, 14, 16, 18, 20, 22, 24, 26, and 28 and capable of binding and inhibiting IL-1. Further, in specific embodiments, the IL-1 antagonist is a modified IL-1 trap comprising one or more receptor components and one or more immunoglobulin-derived components specific for IL-1 and/or an IL-1 receptor. In another embodiment, the IL-1 antagonist is a modified IL-1 trap comprising one or more immunoglobulin-derived components specific for IL-1 and/or an IL-1 receptor.

In certain embodiments, subjects being treated may suffer from chronic neuroinflammation, which may contribute to the neurodegeneration and/or associated cognitive or non-cognitive dysfunction observed in patients with Alzheimer's disease, or any of the other neurodegenerative conditions described herein. In certain embodiments, the subjects being treated with the IL-1 trap of the invention may have improved cognitive or non-cognitive behavioral symptoms following treatment, but will exhibit no change in the amount of beta-amyloid deposited in the brain. In certain embodiments, the subjects being treated with the IL-1 trap of the invention will demonstrate a diminished immune response triggered by the amyloid plaque burden. The reduction in immune response may be shown by a reduction in the number, activated phenotype and/or the size of pen-plaque microglia.

The methods of the invention include administration of the IL-1 antagonist (IL-1 trap) by any means known to the art, for example, subcutaneous, intramuscular, intranasal, intraarterial, intravenous, intracerebral, intraventricular, intrathecal, topical, transvaginal, transdermal, transanal administration or oral routes of administration.

In one embodiment, a therapeutically effective amount of the IL-1 antagonist (IL-1 trap) to be administered to a subject in need thereof ranges from about 1 mg/kg to about 750 mg/kg, or about 10 mg/kg to about 500 mg/kg, or more preferably from about 50 mg/kg to about 150 mg/kg. In one embodiment, the IL-1 trap is administered on a weekly basis.

In one embodiment, a therapeutically effective amount of the IL-1 antagonist (IL-1 trap) to be administered to a subject in need thereof ranges from about 10 mg to about 500 mg, or about 100 mg to about 320 mg. In one embodiment, a therapeutically effective amount of the IL-1 antagonist (IL-1 trap) to be administered to a subject in need thereof is about 100 mg, or about 160 mg, or about 320 mg. In certain embodiments the IL-1 trap is administered on a weekly basis.

In certain embodiments of the therapeutic methods of the invention, the subject is treated with a combination of an IL-1 trap and one or more other (second or third, etc.) therapeutic agents. The other therapeutic agents may be a second IL-1 antagonist, such as, for example, anakinra (KINERET®) or canakinumab, or a second different IL-1 trap, or a recombinant, nonglycosylated form of the human IL-1 receptor antagonist (IL-1Ra), or an anti-IL-18 drug such as IL-18BP or a derivative, an IL-18 Trap, anti-IL-18, anti-IL-18R1, or anti-IL-18Racp. Other co-therapies may include an acetylcholinesterase inhibitor (e.g. ARICEPT® (donepezil HCl), EXELON® (rivastigmine tartrate), RAZADYNE® (galantamine HBr)), or a glutamate pathway modifier, such as, Namenda (memantine HCl). Other co-therapies include aspirin or other NSAIDs, or other inflammatory inhibitors such as inhibitors of caspase-1, p38, IKK1/2, CTLA-4Ig, anti-IL-6 or anti-IL6Ra, etc. Other co-therapies include an antibody specific for tau or an antibody specific for beta amyloid (such as solanezumab, gantenerumab, or bapineuzumab), as well as a microtubule stabilizer (such as epothilone B).

In a third aspect, the invention features a therapeutic method of treating a disease characterized by deposition of beta-amyloid in a subject, or ameliorating at least one symptom of a disease characterized by aberrant deposition of beta-amyloid in a subject, such as Alzheimer's disease, by administering a pharmaceutical composition comprising an IL-1 trap and a pharmaceutically acceptable carrier, in a dose range of about 1 mg/kg to about 300 mg/kg, preferably about 50 mg/kg to about 150 mg/kg alone, or in combination with a second therapeutic agent useful for treating the disease.

In one embodiment, the invention provides for delaying the onset of, or slowing the progression of the disease, comprising administering to a subject in need thereof a pharmaceutical composition comprising an IL-1 antagonist and a pharmaceutically acceptable carrier, in a dose range of about 10 mg/kg to about 300 mg/kg, or about 50 mg/kg to about 150 mg/kg on a weekly basis for a treatment period of between 1 week, 1 month, to one year or more. In certain embodiments, the treatment could last for decades.

In certain embodiments, the IL-1 antagonist to be used as a first or second therapeutic agent is an antibody specific for either IL-1 alpha or IL-1 beta.

In certain embodiments the IL-1 antagonist to be used as a first or second therapeutic agent is a soluble IL-1 receptor that blocks or inhibits the activity of either or both IL-1 alpha and/or IL-1 beta.

In certain embodiments, the IL-1 antagonist to be used as a first or second therapeutic agent is anakinra or canakinumab.

In certain embodiments, the IL-1 antagonist to be used as a first or second therapeutic agent is an IL-1 trap as described herein.

In one particular embodiment, the IL-1 antagonist to be used as a first or second therapeutic agent is the fusion protein (IL-1 trap) as shown in SEQ ID NO: 2, 4, 6, 8, 10, 12,14, 16, 18, 20, 22, 24, 26 or 28. In one embodiment, the IL-1 trap is shown in SEQ ID NO: 28. In one embodiment, the IL-1 trap is shown in SEQ ID NO: 10. The invention encompasses the use of an IL-1 trap substantially identical to the protein of SEQ ID NO: 2, 4, 6, 8, 10, 12,14, 16, 18, 20, 22, 24, 26, 28, that is, a protein having at least 95% identity, at least 97% identity, at least 98% identity to the protein of SEQ ID NO: 2, 4, 6, 8, 10, 12, 14, 16, 18, 20, 22, 24, 26, and 28 and capable of binding and inhibiting IL-1. Further, in specific embodiments, the IL-1 antagonist is a modified IL-1 trap comprising one or more receptor components and one or more immunoglobulin-derived components specific for IL-1 and/or an IL-1 receptor. In another embodiment, the IL-1 antagonist is a modified IL-1 trap comprising one or more immunoglobulin-derived components specific for IL-1 and/or an IL-1 receptor.

In certain embodiments, the IL-1 antagonist may be administered twice a week, or weekly, or monthly, or bi-monthly, or less frequently depending on the results achieved.

In certain embodiments, the doses may be adjusted if it is determined that the patient may need chronic life-long therapy with the IL-1 trap alone, or in conjunction with a second therapeutic agent useful for treating the disease. In certain embodiments, the methods for treating a disease characterized in part by beta amyloid activity or deposition in brain tissue of a patient comprises administering to a subject in need thereof a pharmaceutical composition comprising an IL-1 trap at doses of about 100 mg, or about 160 mg, or about 320 mg and a pharmaceutically acceptable carrier. In certain embodiments the IL-1 trap is administered on a weekly basis. In certain embodiments, the IL-1 trap may be administered on a bi-weekly basis, a monthly basis, or a bi-monthly basis, or less frequently as determined by a patient's response to therapy.

In certain embodiments, the pharmaceutical composition may contain a second or third therapeutically effective amount of another agent useful for treating the disease (a co-formulation). The second or third other therapeutic agent(s) may be a second IL-1 antagonist, such as, for example, anakinra (KINERET®) or canakinumab, a different IL-1 trap, a recombinant, nonglycosylated form of the human IL-1 receptor antagonist (IL-1Ra), or an anti-IL-18 drug such as IL-18BP or a derivative, an IL-18 Trap, anti-IL-18, anti-IL-18R1, or anti-IL-18Racp. Other co-therapies include acetylcholinesterase inhibitors (e.g. ARICEPT® (donepezil HCl), EXELON® (rivastigmine tartrate), RAZADYNE® (galantamine HBr)), or glutamate pathway modifiers, such as, Namenda (memantine HCl). Other co-therapies include aspirin or other NSAIDs, or other inflammatory inhibitors such as inhibitors of caspase-1, p38, IKK1/2, CTLA-4lg, anti-IL-6 or anti-IL6Ra, etc. Other co-therapies include an antibody specific for tau or an antibody specific for beta amyloid (such as solanezumab, gantenerumab, or bapineuzumab), as well as a microtubule stabilizer (such as epothilone B).

In one embodiment, the first and second other therapeutic agent may be administered simultaneously in one pharmaceutical formulation, or may be administered sequentially in different pharmaceutical compositions.

In one embodiment, the disease that is to be treated with a pharmaceutical composition containing an IL-1 trap of the invention is Alzheimer's disease. In certain embodiments, the treatment with the pharmaceutical composition results in preventing the onset of, slowing the progression of, or ameliorating/improving any one or more cognitive or non-cognitive behavioral changes in the subject suffering from Alzheimer's disease, including but not limited to memory loss, inability to learn, depression, anxiety, dementia, inattention, irritability, confusion, mood swings and aggressive and/or apathetic behavior.

A further aspect of the invention provides for the use of an IL-1 antagonist of the invention for treating, or delaying the onset, or the progression of a disease characterized in part by beta amyloid expression, activity, or deposition in a subject in need thereof, or for ameliorating at least one symptom associated with the disease, the method comprising administering to the subject a therapeutically effective amount of an IL-1 antagonist as a first therapeutic agent. In certain embodiments, the IL-1 antagonist may be an antibody or soluble receptor that inhibits the activity of either IL-1 alpha or IL-1 beta, or it may be an IL-1 trap as described herein having the sequences as set forth in SEQ ID NOs: 2, 4, 6, 8, 10, 12,14, 16, 18, 20, 22, 24, 26 or 28. The IL-1 antagonist may be used alone or in conjunction with one or more other therapeutic agents that block, inhibit or antagonize either or both IL-1 alpha or IL-1 beta.

In one embodiment, the IL-1 antagonist is a fusion protein comprising an IL-1 binding portion of the extracellular domain of IL-1 RAcP, an IL-1 binding portion of the extracellular domain of IL-1 R1, and a multimerizing component.

In a related aspect, the invention provides for the use of an IL-1 antagonist of the invention for the preparation of a medicament for treating, or delaying the onset, or the progression of a disease characterized in part by beta amyloid expression, activity, or deposition in a subject in need thereof, or for ameliorating at least one symptom associated with the disease, the method comprising administering to the subject a therapeutically effective amount of an IL-1 antagonist as a first therapeutic agent. In certain embodiments, the IL-1 antagonist may be an antibody specific for IL-1 alpha or IL-1 beta, or it may be a soluble receptor that blocks or inhibits the activity of either IL-1 alpha and/or IL-1 beta, or it may be a fusion protein comprising an IL-1 binding portion of the extracellular domain of IL-1 RAcP, an IL-1 binding portion of the extracellular domain of IL-1 R1, and a multimerizing component.

In one embodiment, the IL-1 fusion protein comprising an IL-1 binding portion of the extracellular domain of IL-1 RAcP, an IL-1 binding portion of the extracellular domain of IL-1 R1, and a multimerizing component is the IL-1 trap shown in any of SEQ ID NOs: 2, 4, 6, 8, 10, 12,14, 16, 18, 20, 22, 24, 26 and 28. In one embodiment, the IL-1 trap is shown in SEQ ID NO: 28. In one embodiment, the IL-1 trap is shown in SEQ ID NO: 10. The invention encompasses the use of an IL-1 trap substantially identical to the protein of SEQ ID NO: 2, 4, 6, 8, 10, 12,14, 16, 18, 20, 22, 24, 26, 28, that is, a protein having at least 95% identity, at least 97% identity, at least 98% identity to the protein of SEQ ID NO: 2, 4, 6, 8, 10, 12, 14, 16, 18, 20, 22, 24, 26, and 28 and capable of binding and inhibiting IL-1. Further, in specific embodiments, the IL-1 antagonist is a modified IL-1 trap comprising one or more receptor components and one or more immunoglobulin-derived components specific for IL-1 and/or an IL-1 receptor. In another embodiment, the IL-1 antagonist is a modified IL-1 trap comprising one or more immunoglobulin-derived components specific for IL-1 and/or an IL-1 receptor.

Other objects and advantages will become apparent from a review of the ensuing detailed description.

### BRIEF DESCRIPTION OF THE FIGURES

FIG. 1a shows the results of the water maze acquisition test.

FIG. 1b shows the results of the water maze retention test (Time in Goal Quadrant).

FIG. 1c shows the results of the water maze retention test (Platform crosses).

FIG. 2 shows the results of the open field test.

FIG. 3 shows increased plaque burden in transgenic animals.

FIG. 4a shows the median number of microglia-like cells per plaque.

FIG. 4b shows the mean size of lba-1-immunoreactive microglial-like cells in transgenic mice stratified by proximity to plaques.

FIG. 4c shows the mean difference in microglial size per animal in microglial in normal tissue (at least 30 microns from the nearest plaque) versus contacting amyloid plaques.

FIG. 5 shows the mean dorsal hippocampal volume at sacrifice for the four groups of animals.

FIG. 6a shows the nucleic acid sequence (SEQ ID NO: 27) of the mouse IL-1 trap and FIG. 6b shows the amino acid sequence (SEQ ID NO: 28) of the mouse IL-1 trap as utilized in the studies described herein.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only the appended claims.

As used in this specification and the appended claims, the singular forms “a”, “an”, and “the” include plural references unless the context clearly dictates otherwise. Thus for example, a reference to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All patents, applications and non-patent publications mentioned in this specification are incorporated herein by reference in their entireties.

### General Description

Alzheimer's disease (AD) is a degenerative brain disorder characterized clinically by both cognitive and non-cognitive behavioral changes, including progressive memory deficits, depression, anxiety, dementia, irritability, mood swings, inattention, aggressive and/or apathetic behavior, confusion, gradual physical deterioration and, ultimately, death. Histologically, the disease is characterized by neuritic plaques, composed primarily of beta amyloid (Aβ) peptide. The plaques are found primarily in the association cortex, limbic system and basal ganglia. Beta amyloid peptide is the cleavage product of beta amyloid precursor protein (β APP or APP). APP is a type I transmembrane glycoprotein that contains a large ectopic N-terminal domain, a transmembrane domain, and a small cytoplasmic C-terminal tail. Alternative splicing of the transcript of the single APP gene on chromosome 21 results in several isoforms that differ in the number of amino acids.

It is believed that Aβ may play a role in the neuropathology of Alzheimer's disease. For example, familial forms of the disease have been linked to mutations in APP and the presenilin genes (Tanzi et al., 1996, Neurobiol. Dis. 3:159-168; Hardy, 1996, Ann. Med. 28:255-258). Furthermore, diseased-linked mutations in these genes result in increased production of the 42-amino acid form of Aβ, the predominant form found in amyloid plaques.

The proinflammatory cytokine interleukin-1 (IL-1) is as an important player in inflammatory processes throughout the body, including in the central nervous system (CNS). IL-1 is found in two distinct isoforms, IL-1α and IL-1β, although IL-1β is considered the primary active isoform. Upregulation of IL-1β is part of the response to a range of CNS insults, including infections, stroke, and traumatic injuries (Allan, S. M., Tyrrell, P. J., & Rothwell, N. J. (2005). Nature Reviews Immunology, 5, 629-640). This neuroinflammatory response is characterized by activation of resident glial cells (microglia and astrocytes), infiltration of peripheral immune cells, and the expression of inflammatory mediators, such as cytokines and chemokines (Shaftel, S. S., Griffin, W. S. T., & O'Banion, M. K. (2008), Journal of Neuroinflammation, 5:7).

IL-1-mediated neuroinflammation may also play a role in the pathogenesis of neurodegenerative diseases (Griffin, W. S., Stanley, L. C., Ling, C., White, L., MacLeod, V., Perrot, L. J., Araoz, C. (1989), Proceedings of the National Academy of Sciences of the United States of America, 86, 7611-7615; Benzing, W. C., Wujek, J. R., Ward, E. K., Shaffer, D., Ashe, K. H., Younkin, S. G., & Brunden, K. R. (1999), Neurobiology of Aging, 20(6), 581-589; Sheng, J. G., Ito, K., Skinner, R. D., Mrak, R. E., Rovnaghi, C. R., Van Eldik, L. J., & Griffin, W. S. (1996), Neurobiology of Aging, 17(5), 761-766).

Although the research to date may support a link between AD and IL-1, it is unclear what role IL-1 plays. IL-1 modulates actions that may contribute to AD pathology, including the synthesis and processing of the Aβ precursor protein and the activity of acetylcholinesterase (Mrak, R. E. & Griffin, W. S. (2001), Neurobiology of Aging, 22(6), 903-908). Chronic IL-1 expression has been associated with demyelination (Ferrari, C. C., Depino, A. M., Prada, F., Muraro, N., Camptbell, S., Podhajcer, O., Pitossi, F. J. (2004), American Journal of Pathology, 165(5), 1827-1837), breakdown of the blood-brain barrier, and neutrophil recruitment (Ferrari et al, supra; Shaftel, S. S., Carlson, T. J., Olschowka, J. A., Kyrkanides, S., Matousek, S. B., & O'Banion, M. K. (2007a), Journal of Neuroscience, 27(35), 9301-9309). IL-1 also activates microglia, which in turn produce pro-inflammatory cytokines such as IL-1, IL-6, and tumor necrosis factor alpha (TNFa). Neuronal insults, such as the accumulation of Aβ, may therefore induce a self-propagating cycle of cytokine activation in which levels of IL-1 constantly rise, leading to neuronal damage and further plaque deposition (Griffin, W. S. T., Sheng, J. G., Royston, M. C., Gentelman, S. M., McKenzie, J. E., Graham, D. I., Mrak, R. E. (1998), Brain Pathology, 8, 65-72).

On the other hand, some have argued that IL-1 plays a beneficial role in AD. Sustained IL-1β overexpression for 4 weeks reduces amyloid plaque expression in swAPP-PS1 mice, a mouse model of Alzheimer's-like pathology that uses the Swedish pedigree mutation in the amyloid precursor protein and the high Alzheimer's risk polymorphism in presenilin-1 (Shaftel, S. S., Kyrkanides, S., Olschowka, J. A., Miller, J. H., Johnson, R. E., & O'Banion, M. K. (2007b), Journal of Clinical Investigation, 117(6), 1595-1604). In the AD brain, microglia expressing IL-1 surround amyloid plaque deposits, suggesting an attempt at phagocytic removal of the plaques (Griffin, W. S., Stanley, L. C., Ling, C., White, L., MacLeod, V., Perrot, L. J., Araoz, C. (1989), Proceedings of the National Academy of Sciences of the United States of America, 86, 7611-7615). In early stages of the disease, microglial activation does seem to delay the progression of AD-like pathology (El Khoury, J., & Luster, A. D. (2008), Trends in Pharmacological Sciences, 29, 626-632; Simard, A. R., Soulet, D., Gowing, G., Julien, J., & Rivest, S. (2006), Neuron, 49, 489-502). However, amyloid plaque burden eventually increases, despite continued microglial activation. One explanation is that the microglia become defective and lose their Aβ-clearing effectiveness. The expression of microglial Aβ receptors and Aβ-degrading enzymes start to decrease around 8 months of age in swAPP-PS1 mice, resulting in reduced Aβ uptake and clearance (Hickman, S. E., Allison, E. K., & El Khoury, J. (2008), Neurobiology of Disease, 28, 8354-8360). The microglia, however, maintain production of IL-1β and TNFα.

Hippocampally-mediated memory processes may be impaired by the overexpression of IL-1 (Moore, A. H., Wu, M., Shaftel, S., Graham, K. A., & O'Banion, M. K. (2009), Neuroscience, 164, 1484-1495; Tanaka, S., Ide, M., Shibutani, T., Ohtaki, H., Numazawa, S., Shioda, S., & Yoshida, T. (2006), Journal of Neuroscience Research, 83, 557-566; Depino, A. M., Alonso, M., Ferrari, C., del Ray, A., Anthony, D., Besedovsky, H., Pitossi, F. (2004), Hippocampus, 14, 526-535).

Although prior research has explored the use of anti-inflammatory drugs as a therapeutic strategy against AD, randomized clinical trials have failed to consistently support the therapeutic effectiveness of NSAIDs against AD (Scharf, S., Mander, A., Ugoni, A., Vajda, F., & Christophidis, N. (1999), Neurology, 53(1), 197-201; Aisen, P. S., Schafer, K. A., Grundman, M., Pfeiffer, E., Sano, M., Davis, K. L., Thal, L. J. (2003), Journal of the American Medical Association, 289(21), 2819-2826).

To date, there have been no studies examining the effects of chronic, systemic IL-1 inhibition on AD-like behavior and pathology. The current studies, described herein, utilized a mouse IL-1 Trap (mlL-1 Trap), an immunoadhesin consisting of a forced IL-1 receptor 1 homodimer fused to a mouse Fc fragment. This trap binds IL-1 at a high affinity, preventing it from binding to its endogenous receptor, and therefore serves as an antagonist of IL-1 signaling. After five months of subcutaneous administration of the mlL-1 Trap in the swAPP-PS1 mutant mouse model of Alzheimer's-like pathology, the effect of IL-1 inhibition on spatial memory, open field activity, amyloid plaque burden, microglial activation, and overall inflammation was examined.

### Definitions

By the term “blocker”, “inhibitor”, or “antagonist” is meant a substance that retards or prevents a chemical or physiological reaction or response. Common blockers or inhibitors include but are not limited to antisense molecules, antibodies, antagonists and their derivatives. More specifically, an example of an IL-1 blocker or inhibitor is an IL-1 antagonist including, but not limited to, an antibody (human or humanized), or an antigen binding portion thereof, to IL-1 alpha and/or IL-1 beta, a soluble IL-1 receptor that blocks or inhibits the activity of either IL-1 alpha or IL-1 beta or both, or an IL-1 trap as described herein, which binds and inhibits IL-1 activity. The relevant IL-1 traps that may be used in the methods of the invention include any of the amino acid sequences noted in SEQ ID NOs: 2, 4, 6, 8, 10, 12, 14, 16, 18, 20, 22, 24, 26 and 28.

By the term “therapeutically effective dose” is meant a dose that produces the desired effect for which it is administered. The exact dose will depend on the purpose of the treatment, and will be ascertainable by one skilled in the art using known techniques (see, for example, Lloyd (1999) The Art, Science and Technology of Pharmaceutical Compounding).

By the term “substantially identical” is meant a protein sequence having at least 95% identity to an amino acid sequence selected from the group consisting of the amino acid sequences SEQ ID NOs: 2, 4, 6, 8, 10, 12, 14, 16, 18, 20, 22, 24, 26 and 28, and capable of binding IL-1 and inhibiting the biological activity of IL-1.

The term “identity” or “homology” is construed to mean the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N- or C-terminal extensions nor insertions will be construed as reducing identity or homology. Methods and computer programs for the alignment are well known in the art. Sequence identity may be measured using sequence analysis software (e.g., Sequence Analysis Software Package, Genetics Computer Group, University of Wisconsin Biotechnology Center, 1710 University Ave., Madison, Wis. 53705). This software matches similar sequences by assigning degrees of homology to various substitutions, deletions, and other modifications.

The term “treating” (or “treat” or “treatment”) refers to processes involving a slowing, interrupting, inhibiting, arresting, controlling, stopping, reducing, ameliorating, or reversing the progression, duration, or severity of an existing symptom, disorder, condition, or disease, but does not necessarily involve a total elimination of all disease-related symptoms, conditions, or disorders through use of the IL-1 trap as described herein. Furthermore, “treating”, “treatment” or “treat” refers to an approach for obtaining beneficial or desired results including clinical results, which include, but are not limited to, one or more of the following: inhibiting, delaying or preventing the onset of, or the progression of, a disease associated with beta amyloid activity, or characterized by aberrant deposition of beta amyloid in a subject, such as in Alzheimer's disease; or inhibiting, preventing, or ameliorating at least one symptom associated with a disease associated with beta amyloid activity, or characterized by aberrant deposition of beta amyloid in a subject, such as in Alzheimer's disease, wherein the symptoms include, but are not limited to, cognitive impairment, memory loss, depression, anxiety, dementia, irritability, confusion, mood swings, aggressive and/or apathetic behavior. “Treatment” or “treating”, as used herein, also refers to increasing the quality of life of those suffering from the disease, decreasing the dose of other medications required to treat the disease and/or prolonging survival of patients.

“Delaying the onset of” Alzheimer's disease or a symptom thereof means to defer, hinder, slow, retard, stabilize, and/or postpone development of the disease, or a symptom associated with, or resulting from the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of Alzheimer's disease is a method that reduces probability of disease development in a given time frame and/or reduces extent of the disease in a given time frame, when compared to not using the method. Such comparisons are typically based on clinical studies, using a statistically significant number of subjects.

“Development” of Alzheimer's disease means the onset and/or progression of Alzheimer's disease within an individual. Alzheimer's disease development can be detectable using standard clinical techniques. However, development also refers to disease progression that may be initially undetectable. For purposes of this invention, progression refers to the biological course of the disease state, in this case, as determined by a standard neurological examination, patient interview, or may be determined by more specialized testing. A variety of these diagnostic tests include, but are not limited to, neuroimaging, detecting alterations of levels of specific proteins in the serum or cerebrospinal fluid (e.g., amyloid peptides and Tau), computerized tomography (CT), positron emission tomography (PET), and magnetic resonance imaging (MRI). “Development” includes occurrence, recurrence, and onset. As used herein “onset” or “occurrence” of Alzheimer's disease includes initial onset and and/or recurrence.

The term “Alzheimer's Disease” or “AD” generally refers to a clinical entity that typically presents with a characteristic progressive amnestic disorder with subsequent appearance of other cognitive, behavioral and neuropsychiatric changes that impair social function and activities of daily living. The initial presentation can be atypical, with non-amnestic focal cortical cognitive symptoms. Disease onset and/or progression can now be assessed through the use of validated and disease-specific biomarkers. Laboratory and neuroimaging biomarkers are highly correlated with neuropathological lesions of AD. These biomarkers can be divided into pathophysiological and topographical markers. Pathophysiological markers correspond to the two etiological degenerative processes that characterize Alzheimer's pathology: the amyloidosis path to neuritic plaques and the tauopathy path to neurofibrillary tangles. They include CSF measurements of concentrations of amyloid beta, total tau, and phosphotau, amyloid PET scanning with Pittsburgh compound B or other radioligands (florbetaben, ¹⁸F-AV-45, etc.). Topographical markers are used to assess the less specific and downstream brain changes that correlate with the regional distribution of AD pathology and include medial temporal lobe atrophy (as measured by MRI) and reduced glucose metabolism in temporo-parietal regions on fluorodeoxyglucose PET. These MRI and PET markers have been shown to predict the development of AD dementia in mild cognitive impairment (MCI) cohorts and to correlate with disease severity. Patients with clinical AD suffer from moderate to severe cognitive and memory impairments that meet the diagnostic criteria of AD and impact work and relationships (including, potentially activities of daily living) and these symptoms are usually accompanied by positive findings on a biomarker tests as described above.

“Prodromal Alzheimer's disease”, also referred to as “predementia stage of AD” refers to the early symptomatic predementia phase of AD in which 1) clinical symptoms including episodic memory loss of the hippocampal type are present, but not sufficiently severe to affect instrumental activities of daily living and do not warrant a diagnosis of dementia; and in which 2) biomarker evidence from CSF or imaging is supportive of the presence of AD pathological changes.

“Preclinical Alzheimer's disease”, which includes both “asymptomatic at-risk state for AD” and “presymptomatic AD” refer to the long asymptomatic stage between the earliest pathogenic events/brain lesions of AD and the first appearance of specific cognitive changes. The “asymptomatic at-risk” state for AD is identified in vivo by evidence of amyloidosis in the brain (with retention of specific PET amyloid tracers) or in the CSF (with changes in amyloid beta, tau, and phosphotau concentrations). “Presymptomatic AD” applies to individuals who will develop AD and this can only be ascertained in families that are affected by rare autosomal dominant monogenic mutations (monogenic AD).

“Cerebral amyloid angiopathy” (CAA), also known as congophilic angiopathy is a form of angiopathy in which amyloid deposits form in the walls of the blood vessels of the central nervous system. The term congophilic is used because the presence of the abnormal aggregations of amyloid can be demonstrated by microscopic examination of brain tissue after application of a special stain called Congo red. The amyloid material is only found in the brain and as such the disease is not related to other forms of amyloidosis. CAA has been identified as occurring either sporadically (generally in elderly populations) or in familial forms such as Flemish, Iowa, and Dutch types. Sporadic forms of CAA have been further characterized into two types based on deposition of amyloid β-protein (Aβ) in cortical capillaries. In all cases, it is defined by the deposition of Aβ in the leptomeningal and cerebral vessel walls. Amyloid deposition predisposes these blood vessels to failure, increasing the risk of a hemorrhagic stroke. Since this can be caused by the same amyloid protein that is associated with Alzheimer's dementia, such brain hemorrhages are more common in people who suffer from Alzheimer's, however they can also occur in those who have no history of dementia. The hemorrhage within the brain is usually confined to a particular lobe and this is slightly different compared to brain hemorrhages that occur as a consequence of high blood pressure (hypertension), a more common cause of a hemorrhagic stroke (or cerebral hemorrhage).

### IL-1- Specific Antagonists

The invention provides IL-1 antagonists for the treatment of diseases characterized by aberrant deposition of beta amyloid in a subject, such as in patients suffering from Alzheimer's disease (AD). In certain embodiments the IL-1 antagonists may include an antibody (or an antigen binding fragment thereof) specific for IL-1 alpha or IL-1 beta, or a soluble receptor that blocks or inhibits the activity of IL-1 alpha or IL-1 beta or both. In certain embodiments, the IL-1 antagonist may be anakinra or canakinumab. In certain embodiments, the IL-1 antagonist may be an IL-1-specific fusion protein antagonist (sometimes referred to as an “IL-1 trap”), which is useful for treating such conditions. IL-1 traps are multimers of fusion proteins containing IL-1 receptor components and a multimerizing component capable of interacting with the multimerizing component present in another fusion protein to form a higher order structure, such as a dimer. Cytokine traps include two distinct receptor components that bind a single cytokine, resulting in the generation of antagonists with dramatically increased affinity over that offered by single component reagents. In fact, the cytokine traps that are described herein are among the most potent cytokine blockers ever described. Briefly, the cytokine traps called IL-1 traps are comprised of the extracellular domain of human IL-1 R Type I (IL-1RI) or Type II (IL-1RII) followed by the extracellular domain of human IL-1 Receptor Accessory protein (IL-1RAcP), followed by a multimerizing component. In one embodiment, the multimerizing component is an immunoglobulin-derived domain, such as, for example, the Fc region of human IgG, including part of the hinge region, the CH2 and CH3 domains. An immunoglobulin-derived domain may be selected from any of the major classes of immunoglobulins, including IgA, IgD, IgE, IgG and IgM, and any subclass or isotype, e.g. IgG1, IgG2, IgG3 and IgG4; IgA-1 and IgA-2. For a more detailed description of the IL-1 traps, see WO00/18932, U.S. Pat. No. 6,927,044; U.S. Pat. No. 7,459,426, which publications are herein specifically incorporated by reference in their entirety. Preferred IL-1-specific fusion proteins have the amino acid sequence shown in SEQ ID NOs: 2, 4, 6, 8, 10, 12, 14, 16, 18, 20, 22, 24, 26 and 28, or a substantially identical protein at least 95% identity to a sequence of SEQ ID NO:4, 6, 8, 10, 12, 14, 16, 18, 20, 22, 24, 26, or 28, and capable of binding and inhibiting IL-1.

In certain embodiments, the IL-1 antagonist comprises an antibody fragment capable of binding IL-1a, IL-β, IL-1R1 and/or IL-1RAcp, or a fragment thereof. The preferred embodiment would be an antagonist of IL-1 R. One embodiment of an IL-1 antagonist comprises one or more antibody fragments, for example, single chain Fv (scFv), is described in U.S. Pat. No. 6,472,179, which publication is herein specifically incorporated by reference in its entirety. In all of the IL-1 antagonist embodiments comprising one or more antibody-derived components specific for IL-1 or an IL-1 receptor, the components may be arranged in a variety of configurations, e.g., a IL-1 receptor component(s)-scFv(s)-multimerizing component; IL-1 receptor component(s)-multimerizing component-scFv(s); scFv(s)-IL-1 receptor component(s)-multimerizing component, ScFv-ScFv-Fc, etc., so long as the molecule or multimer is capable of inhibiting the biological activity of IL-1.

### Anti-IL-1 Human Antibodies and Antibody Fragments

In another embodiment of the IL-1 antagonist useful in the method of the invention, examples of anti-IL-1 antibodies are disclosed in U.S. Pat. No. 4,935,343; U.S. Pat. No. 5,681,933; WO 95/01997; EP 0267611, U.S. Pat. No. 6,419,944; WO 02/16436 and WO 01/53353. The IL-1 antagonist of the invention may include an antibody or antibody fragment specific for an IL-1 ligand (e.g., IL-1α or IL-1β) and/or an IL-1 receptor (e.g., IL-1R1 and/or IL-1RAcp). Antibody fragments include any fragment having the required target specificity, e.g. antibody fragments either produced by the modification of whole antibodies (e.g. enzymatic digestion), or those synthesized de novo using recombinant DNA methodologies (scFv, single domain antibodies or dAbs, single variable domain antibodies) or those identified using human phase display libraries (see, for example, McCafferty et al. (1990) Nature 348:552-554). Alternatively, antibodies can be isolated from mice producing human or human-mouse chimeric antibodies using standard immunization and antibody isolation methods, including but not limited to making hybridomas, or using B cell screening technologies, such as SLAM. Immunoglobulin binding domains also include, but are not limited to, the variable regions of the heavy (V_{H}) or the light (V_{L}) chains of immunoglobulins. Or by immunizing people and isolating antigen positive B cells and cloning the cDNAs encoding the heavy and light chain and coexpressing them in a cell, such as CHO.

The term “antibody” as used herein refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant regions, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Within each IgG class, there are different isotypes (eg. IgG₁, IgG₂, IgG₃, IgG₄). Typically, the antigen-binding region of an antibody will be the most critical in determining specificity and affinity of binding.

An exemplary immunoglobulin (antibody) structural unit comprises a tetramer. Each tetramer is composed of two identical pairs of polypeptide chains, each pair having one light chain (about 25 kD) and one heavy chain (about 50-70 kD). The N-terminus of each chain defines a variable region of about 100-110 or more amino acids primarily responsible for antigen recognition. The terms “variable light chain” (V_{L}) and variable heavy chain (V_{H}) refer to these light and heavy chains respectively.

Antibodies exist as intact immunoglobulins, or as a number of well-characterized fragments produced by digestion with various peptidases. For example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology.

Methods for preparing antibodies are known to the art. See, for example, Kohler & Milstein (1975) Nature 256:495-497; Harlow & Lane (1988) Antibodies: a Laboratory Manual, Cold Spring Harbor Lab., Cold Spring Harbor, N.Y.). The genes encoding the heavy and light chains of an antibody of interest can be cloned from a cell, e.g., the genes encoding a monoclonal antibody can be cloned from a hybridoma and used to produce a recombinant monoclonal antibody. Monoclonal antibodies can be humanized using standard cloning of the CDR regions into a human scaffold. Gene libraries encoding human heavy and light chains of monoclonal antibodies can also be made from hybridoma or plasma cells. Random combinations of the heavy and light chain gene products generate a large pool of antibodies with different antigenic specificity. Techniques for the production of single chain antibodies or recombinant antibodies (U.S. Pat. No. 4,946,778; U.S. Pat. No. 4,816,567) can be adapted to produce antibodies used in the fusion proteins and methods of the instant invention. Also, transgenic mice, or other organisms such as other mammals, may be used to express human, human-mouse chimeric, or humanized antibodies. Alternatively, phage display technology can be used to identify human antibodies and heteromeric Fab fragments that specifically bind to selected antigens.

### Antibody Screening and Selection

Screening and selection of preferred antibodies can be conducted by a variety of methods known to the art. Initial screening for the presence of monoclonal antibodies specific to a target antigen may be conducted through the use of ELISA-based methods, for example. A secondary screen is preferably conducted to identify and select a desired monoclonal antibody for use in construction of the multi-specific fusion proteins of the invention. Secondary screening may be conducted with any suitable method known to the art. One preferred method, termed “Biosensor Modification-Assisted Profiling” (“BiaMAP”) is described in co-pending U.S. Ser. No. 60/423,017 filed 1 Nov. 2002, herein specifically incorporated by reference in its entirety. BiaMAP allows rapid identification of hybridoma clones producing monoclonal antibodies with desired characteristics. More specifically, monoclonal antibodies are sorted into distinct epitope-related groups based on evaluation of antibody:antigen interactions. Antibodies capable of blocking either a ligand or a receptor may be identified by a cell based assay, such as a luciferase assay utilizing a luciferase gene under the control of an NFKB driven promoter. Stimulation of the IL-1 receptors by IL-1 ligands leads to a signal through NFKB thus increasing luciferase levels in the cell. Blocking antibodies are identified as those antibodies that blocked IL-1 induction of luciferase activity.

### Treatment Population

The therapeutic methods of the invention are useful for treating individuals affected with a disease or condition characterized by aberrant deposition and/or activity of beta amyloid in a subject. Diseases or conditions for which the current IL-1 antagonists may be used include Alzheimer's disease, multi-infarct dementia, cognitive impairment and cerebral amyloid angiopathy (CAA). The stages of Alzheimer's disease (AD) or cerebral amyloid angiopathy (CAA) for which the treatments may be effective include any of the following: prodromal AD or CAA, preclinical AD or CAA and clinical stage AD or CAA, as described previously.

### Therapeutic Administration and Formulations

The invention provides therapeutic compositions comprising the IL-1 antagonist (IL-1 trap) of the present invention. The administration of therapeutic compositions in accordance with the invention will be administered via a suitable route including, but not limited to, intravenously, subcutaneously, intramuscularly, intrathecally, intracerebrally, intraventricularly, intranasally, with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of the IL-1 antagonist, e.g. IL-1 trap, may vary depending upon the age and the size of a subject to be administered, target disease, conditions, route of administration, and the like. When the antibody of the present invention is used for treating Alzheimer's disease, it is advantageous to intravenously administer the antibody of the present invention normally at a single dose of about 1 to about 750 mg/kg body weight, more preferably about 5 to about 300, about 10 to about 200, or about 50 to about 150 mg/kg body weight. Depending on the severity of the condition, the frequency and the duration of the treatment can be adjusted. In certain embodiments, the IL-1 trap of the invention can be administered as an initial dose of at least about 0.1 mg to about 800 mg, about 1 to about 500 mg, about 5 to about 300 mg, or about 10 to about 200 mg, to about 100 mg, or to about 50 mg. In certain embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks. The IL-1 trap of the invention may be administered to a subject using any of the above described dosing regimens throughout the life of the patient.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, transdermal, intramuscular, intraperitoneal, intravenous, subcutaneous, intranasal, epidural, intrathecal, intraventricular, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The pharmaceutical composition can be also delivered in a vesicle, in particular a liposome (see, for example, Langer (1990) Science 249:1527-1533).

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, Inn.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but certainly are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousands Oaks, Calif.), the PENLET™ (Haselmeier, Stuttgart, Germany), the EPIPEN (Dey, L.P.) and the HUMIRA™ Pen (Abbott Labs, Abbott Park, Ill.), to name only a few.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 750 mg per dosage form in a unit dose; especially in the form of injection, it is preferred that the aforesaid antibody is contained in about 5 to about 100 mg and in about 10 to about 250 mg for the other dosage forms.

### Combination Therapies

In numerous embodiments, the IL-1 antagonists of the present invention may be administered in combination with one or more additional compounds or therapies. Combination therapy may be simultaneous or sequential. The IL-1 antagonists of the invention may be combined with other IL-1 antagonists, such as antibodies specific for IL-1 alpha or IL-1 beta (or antigen binding fragments thereof), a soluble IL-1 receptor, or other different IL-1 traps. The IL-1 traps of the invention may also be be combined with, for example, ARICEPT® (donepezil HCl), EXELON® (rivastigmine tartrate), RAZADYNE® (galantamine HBr), steroids, anakinra (KINARET®, Amgen) or canakinumab. The IL-1 traps of the invention may also be combined with anti-IL-18 drugs, such as for example, IL-18BP or a derivative, an IL-18 Trap, anti-IL-18, anti-IL-18R1, or anti-IL-18Racp. Other co-therapies include aspirin or other NSAIDs, steroids such as prednisolone, other inflammatory inhibitors such as inhibitors of caspase-1, p38, IKK1/2, CTLA-4lg, anti-IL-6 or anti-IL6Ra, an antibody specific for tau, an antibody specific for beta amyloid (such as solanezumab, gantenerumab, or bapineuzumab) or a microtubule stabilizers (such as epothilone B).

### Administration Regimens

According to certain embodiments of the present invention, multiple doses of the IL-1 trap may be administered to a subject over a defined time course. The methods according to this aspect of the invention comprise sequentially administering to a subject multiple doses of the IL-1 trap of the invention. As used herein, “sequentially administering” means that each dose of the IL-1 trap is administered to the subject at a different point in time, e.g., on different days separated by a predetermined interval (e.g., hours, days, weeks or months). The present invention includes methods, which comprise sequentially administering to the patient a single initial dose of the IL-1 trap, followed by one or more secondary doses of the IL-1 trap, and optionally followed by one or more tertiary doses of the IL-1 trap.

The terms “initial dose,” “secondary doses,” and “tertiary doses,” refer to the temporal sequence of administration of an IL-1 trap of the invention. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the IL-1 trap, but generally may differ from one another in terms of frequency of administration. In certain embodiments, however, the amount of the IL-1 trap contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment of the present invention, each secondary and/or tertiary dose is administered % to 26 (e.g., %, 1, 1%, 2, 2%, 3, 3%, 4, 4%, 5, 5%, 6, 6%, 7, 7%, 8, 8%, 9, 9%, 10, 10%, 11, 11%, 12, 12%, 13, 13%, 14, 14%, 15, 15%, 16, 16%, 17, 17%, 18, 18%, 19, 19%, 20, 20%, 21, 21%, 22, 22%, 23, 23%, 24, 24%, 25, 25%, 26, 26%, or more) weeks after the immediately preceding dose. The phrase “the immediately preceding dose,” as used herein, means, in a sequence of multiple administrations, the dose of the IL-1 trap, which is administered to a patient prior to the administration of the very next dose in the sequence with no intervening doses.

The methods according to this aspect of the invention may comprise administering to a patient any number of secondary and/or tertiary doses of the IL-1 trap. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

### Pharmaceutical Compositions

The present invention also provides pharmaceutical compositions. Such compositions comprise a therapeutically effective amount of an active agent, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In one embodiment, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the active agent of the invention which will be effective in the treatment of Alzheimer's disease can be determined by standard clinical techniques based on the present description. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 20 micrograms to 2 grams of active compound per kilogram body weight. Suitable dosage ranges for intra-nasal administration are generally about 0.01 pg/kg body weight to 1 mg/kg body weight. Effective doses may be extrapolated from dose-response curves derived from in vitro or animal model test systems.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds that are sufficient to maintain therapeutic effect. In cases of local administration or selective uptake, the effective local concentration of the compounds may not be related to plasma concentration. One having skill in the art will be able to optimize therapeutically effective local dosages without undue experimentation.

The amount of compound administered will, of course, be dependent on the subject being treated, on the subject's weight, the severity of the affliction, the manner of administration, and the judgment of the prescribing physician. The therapy may be repeated intermittently while symptoms are detectable or even when they are not detectable. The therapy may be provided alone or in combination with other drugs.

### Kits

The invention also provides an article of manufacturing comprising packaging material and a pharmaceutical agent contained within the packaging material, wherein the pharmaceutical agent comprises at least one IL-1-specific fusion protein of the invention and wherein the packaging material comprises a label or package insert which indicates that the IL-1-specific fusion protein can be used for treating a disease characterized by aberrant deposition of beta amyloid, such as Alzheimer's disease.

Other features of the invention will become apparent in the course of the following descriptions of exemplary embodiments which are given for illustration of the invention and are not intended to be limiting thereof.

### EXAMPLES

The following example is put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Effect of an IL-1 Antagonist (IL-1 Trap) in a Mouse Model of Alzheimer's-Like Pathology

### Methods

### Animals and Injections:

Subjects were 40 male mice split into two cohorts of 20. Within each cohort, ten mice were wild type (WT) animals and ten were tandem transgenic (Tg) for both the Swedish pedigree mutation in the amyloid precursor protein (SwAPP) and the high Alzheimer's risk polymorphism in presenilin-1 (PS-1). Both the wild type controls and the transgenic mice were of the C57B1/6 background. The mice were housed in a temperature-stabilized facility on a 12:12 light:dark cycle (lights on 07:00), with food and water available ad libitum.

Starting at approximately 8 months of age, animals were administered biweekly injections of mouse IL-1 Trap or control mFc subcutaneously. Five Tg animals and five WT animals of each cohort received mlL-1 Trap at a dose of 10 mg/kg. The rest of the mice received mouse Fc (mFc) at the same dose and volume. Mice were weighed weekly in order to establish dosage and evaluate animal health. The injections continued for 5 months. Three mice died before completing the behavioral testing phase (one WT animal receiving mFc, one TG animal receiving mFc and one TG animal receiving mlL-1 Trap.)

Behavioral testing:

Morris Water Maze: During the fifth month of injections, the Morris Water Maze Test was conducted to evaluate spatial learning and memory. A pool measuring 105 cm in diameter and 35cm in depth was filled with water made opaque with non-toxic white paint. The pool was then divided conceptually into four quadrants, one of which contained an escape platform hidden 2.5 cm beneath the surface. Animals were placed in a different, pseudo-randomly selected quadrant at the start of each trial, and the latency to escape the maze onto the hidden platform was measured. There were two trial blocks per day for five days, each consisting of three one-minute trials. Between trials, animals were held with a towel for approximately 30 seconds before being placed back in the water. After each block, they were placed in a holding cage lined with towels until dry and then returned to their home cages. The testing was conducted at the same time each day with a 3-hour interval between the first and second daily blocks. Any animal not locating the platform within one minute was assigned a latency of 60 seconds and guided to the platform by hand before being removed from the water. Normal animals were expected to demonstrate a decrease in latency to escape across trials in the water maze, indicating acquisition of the location of the platform over time.

One hour after the final block of water maze acquisition testing, animals were returned to the maze for a 30 second probe trial with the platform removed to test their retention of the platform's location. Retention was assessed via a measure of the amount of time animals spent in the quadrant that formerly housed the platform, termed the “goal quadrant.” The number of times the animals swam over the spot where the platform was located was also counted. Animals with normal retention are expected to spend more time in the “goal quadrant” than in other quadrants and to make more “platform crosses”, thus demonstrating retention of the spatial location of the escape platform.

For each block, the animals' median latency to escape the maze across the three trials was recorded and these median latencies were used for statistical analysis. In addition, animals' swim speeds were estimated by dividing latency to escape by the number of maze quadrants crossed per trial to calculate mean quadrant crossing time. The median quadrant crossing time for each trial block was used as a covariate in the statistical analysis to account for potential differences in motor speed.

### Open Field:

During the fifth month, the open field test was conducted in order to explore some of the non-cognitive behavioral symptoms of Alzheimer's-like disease and whether IL-1 inhibition has an effect on those symptoms. Specifically, open field can be used to measure general locomotor and exploratory activity (Walsh, R. N. & Cummins, R. A. (1976), Psychological Bulletin, 83(3), 482-504). Animals were placed into a white, box-like apparatus measuring 48 x 48×24 cm. The inside floor was divided into nine grids, each measuring 16×16 cm. Animals were placed in the center grid and allowed to freely explore the apparatus for a six minute trial. The number of total grid crossings was recorded as a measure of general locomotor and exploratory behavior.

### Tissue Collection and Processing:

After 5 months of treatment, the animals were sacrificed and brains were prepared for histological analysis. All animals were overdosed with a pentobarbital-based euthanasia solution and perfusion fixed. Cold heparinized isotonic (0.9%) saline was run through the body to exsanguinate the animal and animals were then perfused with 4% paraformaldehyde first in acetate, and then borate buffer (100 ml each). Upon completion of the perfusion, brains were removed and placed in 30% buffered sucrose for 3-7 days. The fixed brains were sectioned coronally at 50 μm and stored in cryoprotectant (Watson, R. E., Wiegand, S. J., Clough, R. W., & Hoffman, G. E. (1986), Peptides, 7(1), 155-159) at −20° C. until they were stained.

### Histology:

Brain tissue from the animals was used for histological analysis. Sections were stained in a 1:12 series with Congo Red to detect the presence of amyloid plaques and with cresyl violet for Nissl bodies in separate sections for visualization of all cells. Immunostaining was conducted to visualize the microglial marker lba-1 (Millipore rabbit polyclonal anti-lba-1, 1:500). For the second cohort of animals, a double-stain was conducted for both plaques and microglia in order to evaluate parameters of microglia at various distances from plaques.

### Image Analysis:

Amyloid plaque burden was analyzed by contrast analysis using the ImageJ image analysis software program (NIH). A minimum of 2 bilateral sections of hippocampus in a 1:12 series were selected and images were captured in 10× using the PictureFrame program. In each picture, the red color of the stain was isolated and the background faded using Adobe Photoshop to achieve contrast. Identical processing parameters were used for all sections. The entire image was then converted to black and white and imported to Image J. For each image, a set percentage of the background was removed from all images and the image was converted to binary. The percentage of area stained, number of separate plaques, and average size per plaque were recorded for each animal.

The level of overall inflammation was assessed by subjective ratings. An experienced histologist blind to the experimental conditions examined cresyl violet-stained hippocampal sections at a magnification of 40×. Each animal was given a rating of “none,” “mild,” “moderate,” or “marked,” based on the presence of inflammatory cell profiles (microglia, immune cells), with “none” signifying no inflammatory cells and “severe” being the highest amount of inflammation observed. The presence of microglial-invested deposits was also noted. For each animal showing plaque-like deposits, 5 random deposits were selected from the hippocampus and the surrounding microglia-like cells were counted. The median number of microglial profiles per plaque was recorded for each animal. Finally, double stains for congo red-positive plaques and lba-1-immunoreactive microglia were used to measure the sizes of microglia contacting plaques (contact), within 20 microns of a plaque (adjacent), or greater than 30 microns from a plaque (distant).

### Statistical Analysis

For water maze acquisition, the median latency to escape to platform was recorded for each animal for each trial block. A three-way mixed Factorial analysis of variance (ANOVA) was conducted using treatment and genotype as the between groups independent variables and block as a repeated measure. For retention, the proportion of time spent in the goal quadrant was recorded for each animal along with the number of platform crosses. Two-way ANOVAs (genotype×treatment) were conducted with each of these as dependent variables.

To evaluate locomotor activity and exploratory behavior, the number of grid crossings in the open field test was counted for each animal. A two-way factorial ANOVA was conducted using treatment and genotype as the between groups independent variables.

Two-way ANOVAs (genotype×treatment) were conducted for each of the following measures: amyloid plaque total area, plaque number, hippocampal volume, microglial size, and average plaque size. For these measures, values for individual hemispheres were averaged to calculate a mean for each animal. Finally, regression analyses were conducted to assess the predictive relationship between memory performance and plaque pathology for each treatment group (mFc and mlL-1 Trap).

A non-parametric log-linear analysis for qualitative variables was conducted to analyze the overall inflammation ratings. An independent groups t-test was conducted to compare number of microglia-like cells surrounding hippocampal deposits in mFc-treated transgenic mice versus mlL-1-treated trap transgenic mice.

Data are presented as mean across all animals within a treatment/genotype group and standard error of the mean (SEM). Statistical significance was set at an alpha level of 0.05.

### Results

### General Health:

Mice were weighed and evaluated weekly to assess their general health. Out of the original 40 animals, 37 survived to sacrifice. In the first cohort, one transgenic animal in the mFc-treated group died before the behavioral testing stage. From the second cohort, one transgenic animal in the mlL-1 Trap group died before the behavioral testing stage and one wild type animal in the mlL-1 Trap group died after completing some behavioral testing. At the final weighing before perfusion, the transgenic mice weighed about 27% less than the wild type mice (F_{(1,33})=55.46, p<0.001). Although the transgenic mice weighed less, they were actually closer to the normal body weight for adult male mice, and appeared grossly healthy. The mlL-1 Trap had no negative impact on body weights (F_{(1,33})=0.00, p=0.994) and the mice all seemed healthy throughout the study.

### Water Maze:

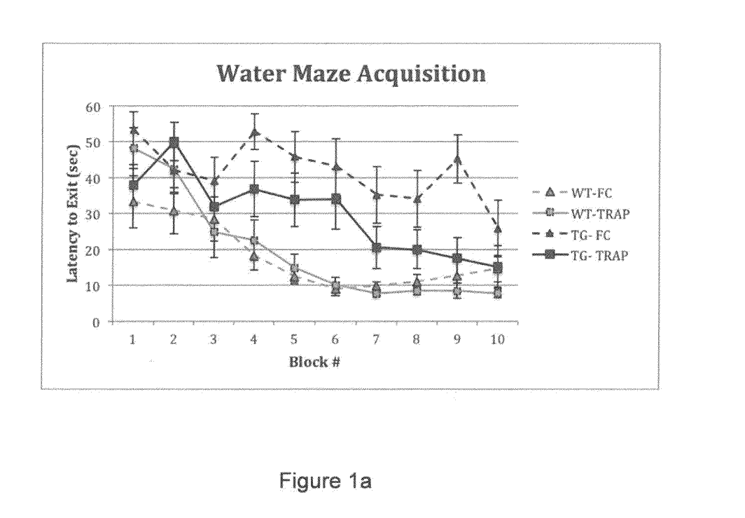

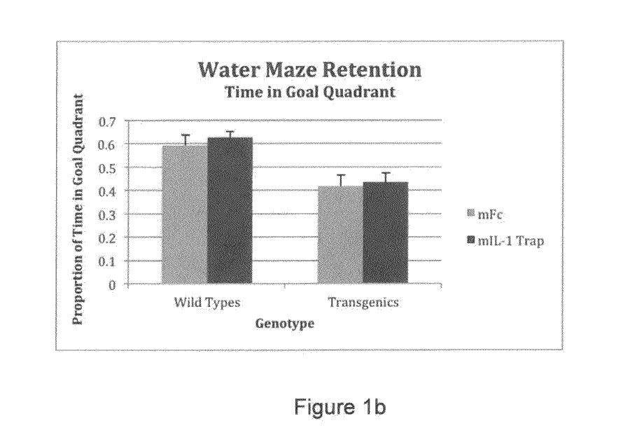

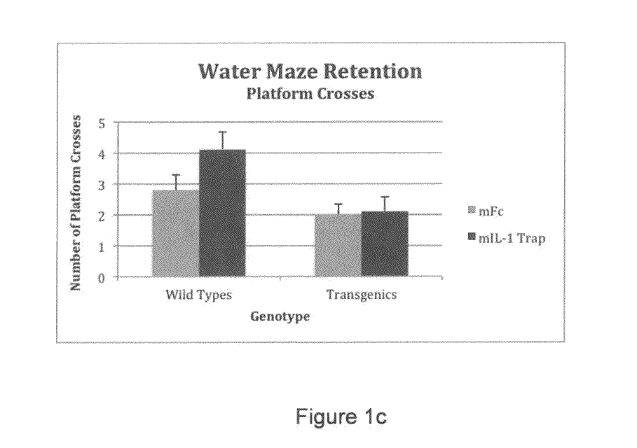

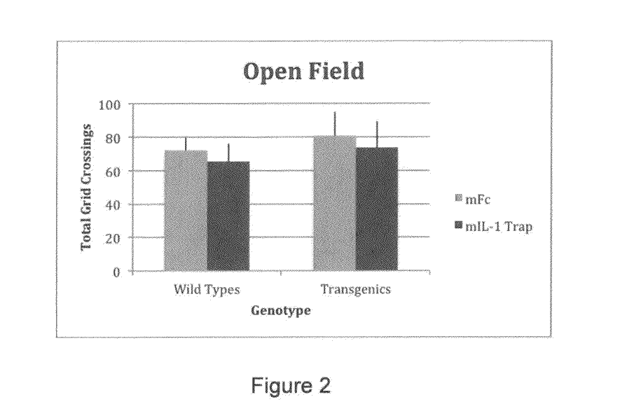

As expected, a three-way mixed Factorial ANOVA (genotype×treatment×trial block) showed that the wild type animals performed significantly better overall than the swAPP/PS-1 double transgenic animals in water maze acquisition (F_{(1,33)}=29.71, p<0.001). The mlL-1 Trap had no significant overall effect on water maze performance (F_{(1,33)}=2.76, p=0.11), but there was a significant interaction between the two factors (F_{(1,33)}=4.61, p=0.039; FIG. 1), such that the trap selectively improved performance in the transgenic animals. An analysis of cohort effects revealed a significant cohort by genotype interaction, such that the transgenic animals of the second cohort performed worse overall than those in the first cohort. However, there was no cohort by treatment effect and no cohort by genotype by treatment effect, indicating that the trap affected the mice similarly across both cohorts.

We also assessed whether differences in swim speed could be influencing these results. Slower swim speeds have been reported in transgenic AD mice (Ying, T., Xu, Y., Scearce-Levie, K., Ptacek, L. J., & Fu, Y. H. (2010), Neurogenetics, 11(1), 41-52), which can interfere with an animal's latency to swim to the platform. A three-way mixed Factorial ANOVA (genotype×treatment×trial block) revealed no significant differences in quadrant crossing time across genotype (F_{(1,33)}=0.61, p=0.442) or treatment (F_{(1,33)}=1.13, p=0.296), and no genotype by treatment interaction (F_{(1,33)}=0.47, p=0.50). In addition, we did not see any significant general locomotor differences in the open field test (see Open Field section), suggesting that motor differences cannot account for the acquisition results observed.

On the retention portion of the water maze, wild type animals spent significantly more time in the goal quadrant (F_{(1,33)}=18.83, p<0.001) and made significantly more platform crosses (F_{(1,33)}=8.86, p=0.005) than transgenic animals, confirming the memory impairment in the transgenics. There was no significant effect of IL-1 Trap treatment on proportion of time spent in the goal quadrant (F_{(1,33)}=0.313, p=0.58) and there was no interaction between the factors (F_{(1,33)}=0.04, p=0.84) (FIG. 1b). There was also no significant effect of treatment on number of platform crosses (F_{(1,33)}=2.29, p=0.14) and no interaction between treatment and genotype (F_{(1,33)}=.1.63, p=0.21) (FIG. 1c).

### Open Field

A two-way ANOVA (genotype×treatment) was conducted to examine differences in locomotor activity and exploratory behavior. Overall, there was no difference in total number of grid crossings between wild type and transgenic animals (F_{(1,33)}=0.699, p=0.409), and no difference between animals treated with mFc and those treated with mll-1 Trap (F_{(1,33)}=0.18, p=0.673). There was no interaction between genotype and treatment (F_{(1,33)}=0.03, p=0.873). These results indicate that transgenic mice did not differ in overall level of locomotor activity compared to wild type mice and that level of activity was not affected by treatment with mlL-1 trap.

### Plaque Burden Analysis

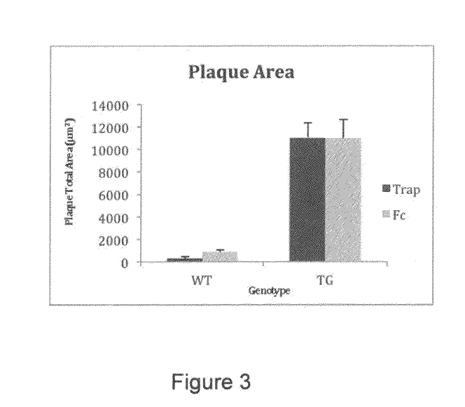

As expected, the plaque burden contrast analysis revealed that swAPP/PS-1 transgenic mice had significantly more plaques (F(1,13)=34.21, p<0.001), more total area covered by plaques (F(1,13)=31.04, p<0.001), and a higher average plaque size (F(1,13)=7.43, p=0.02) than the wild type animals. Indeed, no Congo red-positive plaques were observed in any wild type animal (data not shown). There was no significant difference between animals given mlL-1 Trap and mFc on any of these measures, and no interactions between genotype and treatment (FIG. 3).

We also examined the relationship between plaque burden and spatial learning acquisition on block 5 (halfway through learning) and block 10 (final performance block) of water maze acquisition. For animals given mFc, there was a significant positive correlation between total plaque area and latency to escape on block 5 (r(6)=0.90, p=0.002), such that animals with more plaque coverage took longer to find the platform during the learning trial. The correlation remained significant when wild type animals, which had no plaques, were excluded from analysis (r(2)=0.96, p=0.04). A regression analysis could not be conducted to regress plaques against learning in the wild type animals, because no plaques were detected in any of these animals. The mlL-1 Trap animals had a smaller, non-significant correlation between plaque burden and learning performance (r(7)=0.46, p=0.21), which disappeared completely when wild type animals were excluded from analysis (r(3)=0.004, p=0.99), suggesting that mlL-1 Trap eliminated the association between plaque burden and cognitive performance in the swAPP/PS-1 transgenic mice. By block 10, when animals had reached peak performance, the amount of plaque coverage was no longer significantly correlated with water maze performance for either treatment group. Sample sizes were too small to determine whether the differences in correlations between the two genotypes were statistically significant.

### Inflammation

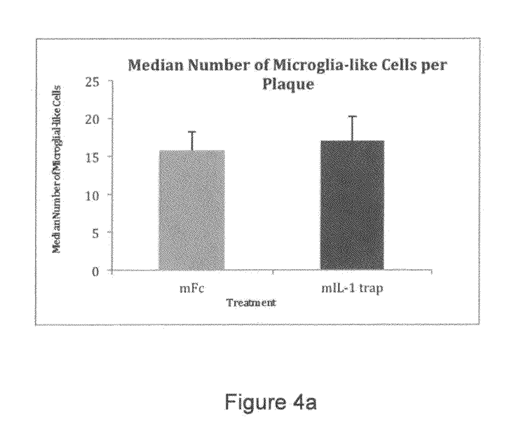

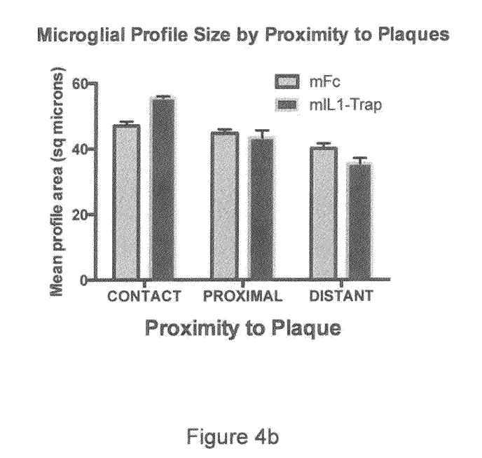

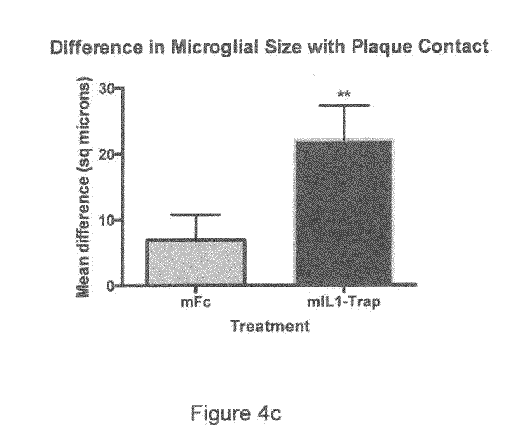

A log-linear analysis of overall subjective inflammation ratings did not reveal a significant difference between mFc and mlL-1 Trap-treated animals (G²(4)=5.22, p=0.26) (Table 1). An independent groups t-test was conducted on the measure of the overall number of microglia-like cells surrounding the plaque-like deposits in the hippocampi of transgenic animals, and there was no significant difference in microglial profile counts between the Fc and mlL-1 Trap-treated animals. (t₍₇₎=−0.30, p=0.78) (FIG. 4a). However, an analysis of microglial profile size showed a statistically significant change in microglial size with IL-1 Trap treatment interacting with distance from plaques (FIG. 4b, interaction F(2,12)=16.127, p=0.0004). Specifically, while microglial sizes were larger for microglia contacting plaques in all animals, the differences in average size for plaques contacting microglia were significantly greater in IL-1 Trap-treated animals. To better illustrate this effect, the difference between the mean size of microglia in unaffected tissue versus plaque-containing tissue for each animal was calculated. This difference in microglial size per animal showed a statistically significant effect of IL-1 Trap on enhancement in microglial size upon plaque contact (FIG. 4c, t(6)=4.864, p=0.0028).

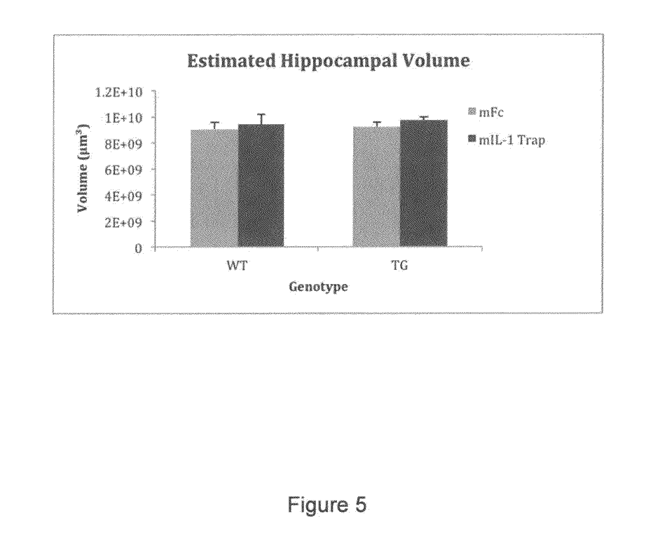

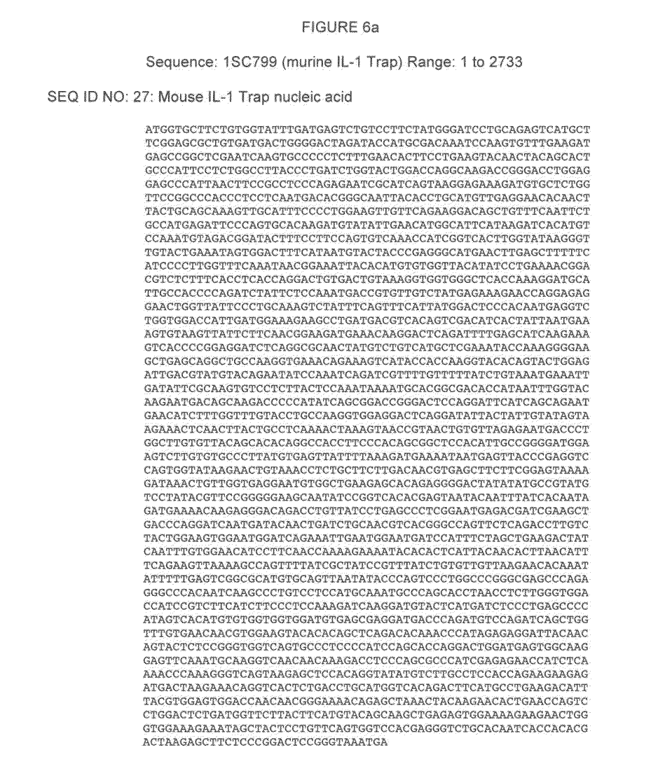

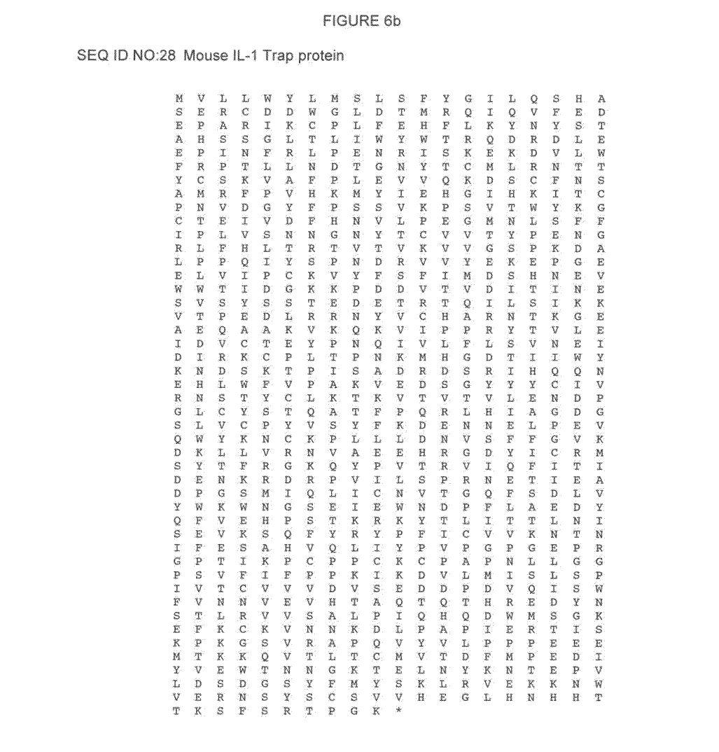

In addition, as shown in FIG. 5, this model of disease produces no significant effect on overall hippocampal volume (F(1,13)=0.236, p=0.635), nor does IL-1 Trap impact hippocampal volume in either the wild type or transgenic mice (treatment F(1,13)=0.800, p=0.387; interaction F(1,13)=0.015, p-0.903).

TABLE 1
Distribution of Overall Inflammation Ratings

| | | | |
|---|---|---|---|
| None | Mild | Moderate | Marked |

WT

| | | | | |
|---|---|---|---|---|
| mFc | 40% | 60% | 0% | 0% |
| mIL-1 Trap | 20% | 20% | 60% | 0% |

TG

| | | | | |
|---|---|---|---|---|
| mFc | 0% | 25% | 25% | 50% |
| mIL-1 Trap | 20% | 50% | 20% | 20% |

### Discussion

Chronic neuroinflammation, which includes elevated IL-1 expression, is a prominent feature of Alzheimer's disease, which may contribute to the neurodegeneration and associated cognitive dysfunction observed in patients with Alzheimer's Disease. In this study, systemic administration of the mlL-1 Trap was used to inhibit interleukin-1 signaling for 5 months after the onset of disease in order to observe its effects on both behavior and Alzheimer's-like brain pathology. The study was done to determine if IL-1 inhibition would improve performance on measures of learning and memory while reducing amyloid plaque burden. Although the mlL-1 Trap did improve water maze performance in transgenic mice, it did not significantly alter amyloid plaque burden. It did, however, increase the size of microglia contacting amyloid plaques while decreasing the size of microglia overall in the brain, suggesting that although it didn't alter the amount of beta-amyloid deposited in the transgenic brains, it may have altered the nature of the immune response triggered by the plaques. In addition, mlL-1 Trap completely eliminated the significant positive relationship between plaque burden and cognitive impairments, showing that although the plaques were present, they were no longer associated with cognitive impairments in the animals.

### Water Maze

Our finding that the mlL-1 Trap selectively improved water maze performance for transgenic animals shows that IL-1 inhibition can slow the cognitive decline resulting from overexpression of mutant Presenilin-1 and the Swedish APP mutation. In the present study, IL-1 inhibition improved acquisition (e.g. learning) while having no effect on retention (e.g. memory), showing that the treated mice may have used compensatory strategies to assist with their performance of the task.

### Open Field

The open field test is used to evaluate locomotor and exploratory behaviors in rodents. Results in swAPP-PS1 transgenic mice showed no significant difference in locomotor activity compared to wild type mice in a six-minute open field test. The lack of significant differences between groups on both open field and on water maze swim speed suggest that differences in learning and memory performance cannot be attributed to alterations in motor or exploratory behavior.

### Amyloid Plaques

In addition to the behavioral testing as a measure of Alzheimer's like pathology, a Congo Red stain was performed for the detection of amyloid plaques. We hypothesized that chronic IL-1 inhibition would result in reduced amyloid plaque pathology. However, the mlL-1 Trap did not significantly decrease hippocampal plaque burden in the transgenic animals.

The water maze data taken together with the plaque analysis provides an interesting look at the nature of AD-like pathology in the swAPP-PS1 transgenic mice. If, as our data indicate, the Trap did improve spatial memory in these mice without reducing the amount of amyloid plaques in the brain, it would imply that factors other than β-amyloid deposition are contributing to their cognitive deficits. One explanation is that the mlL-1 Trap may be improving memory by inhibiting the immune response to the plaques without reducing the plaques themselves.

To further evaluate the role of amyloid plaques, we looked at the relationship between plaque deposits and water maze performance for transgenic animals treated with mFc and for animals treated with mlL-1 Trap. In this study, transgenic AD animals given mFc showed a very strong significant correlation (r=0.96) between plaque burden and water maze acquisition performance during spatial memory learning (block 5), such that animals with more amyloid deposition took longer to find the platform. However, animals given mlL-1 Trap showed no correlation (r=0.004) between plaque burden and the same water maze measure. The fact that these results were obtained with such small sample sizes is especially promising, although further studies with larger sample size are warranted. If the mlL-1 Trap, which suppresses part of the neuroinflammatory response, actually eliminates the relationship between amyloid plaques and memory, it would support the hypothesis that it is the immune response to the plaques, rather than the plaques themselves, that are causing the memory impairments.

### Inflammation

In order to probe the possibility that mlL-1 Trap changed the immune response to the plaques, a subjective analysis of the overall level of inflammation around the plaques in the hippocampi of the transgenic animals was conducted. The initial analysis involved the assignment of an overall subjective rating of peri-plaque inflammation by an experienced histologist blind to animal treatment. This analysis did not reveal any significant overall differences in inflammatory cell investment around the plaques. In support of this finding, a quantitative count of one specific inflammatory cell type, the resident brain macrophages (microglia), revealed no difference in overall microglial count per plaque. However, a statistical trend was observed toward a significant difference in microglial size between transgenic animals treated with IL-1 Trap versus mFc control. The differences that were observed included a shift toward smaller microglia at rest, but larger microglia contacting plaques. Although more research will be necessary to fully interpret this trend, one possibility is that microglia are less activated basally in IL-1 Trap treated animals, but are more phagocytic when confronted with pathological deposits. While we do not have direct evidence that IL-1 Trap changed the activation subtype of microglia, this pattern of sizes may indicate that more M2 than M1 microglia were present in the brains of IL-1 Trap-treated animals. M2 microglia tend to be less activated overall, but have more potential for phagocytosis.

It is possible that the inhibition of interleukin 1 in the present study is affecting some of the detrimental microglial processes, without improving their plaque-clearing ability. Further morphometric and neurochemical analysis will help determine the full extent of changes in microglial activation and morphology.

### Summary

The current study showed a potentially protective role of systemic mlL-1 Trap treatment in the swAPP/PS-1 double transgenic model of Alzheimer's Disease. In particular, the current study provides support for the growing hypothesis that amyloid plaques do not, in themselves, underlie the hallmark cognitive impairments of Alzheimer's Disease. Indeed, it supports the idea that components of the inflammatory cascade, perhaps triggered in part by the presence of the plaques, are major pathogenic contributors. In addition, our data provide initial proof of concept for the potential use of IL-1 inhibition to treat the cognitive impairments of Alzheimer's Disease. Finally, our data show that even large biological inhibitors of IL-1, such as the IL-1 traps described herein, given after disease onset, could provide significant benefit to patients suffering from the profound cognitive impairments characterizing this devastating disease.

1. A method for treating, or delaying the onset, or the progression of a disease characterized in part by beta amyloid expression, activity, or deposition in a subject in need thereof, or for ameliorating at least one symptom associated with the disease, the method comprising administering to the subject a therapeutically effective amount of an IL-1 antagonist as a first therapeutic agent, wherein the IL-1 antagonist is selected from the group consisting of an antibody specific for IL-1 alpha or IL-1 beta, or an antigen-binding fragment thereof, a soluble IL-1 receptor, and an IL-1 trap, wherein the IL-1 trap is a fusion protein comprising an IL-1 binding portion of the extracellular domain of IL-1RAcP, an IL-1 binding portion of the extracellular domain of IL-1R1, and a multimerizing component.

2. A method for treating, or delaying the onset, or the progression of a disease characterized in part by beta amyloid expression, activity, or deposition in a subject in need thereof, or for ameliorating at least one symptom associated with the disease, the method comprising administering to the subject a therapeutically effective amount of an IL-1 antagonist as a first therapeutic agent, wherein the IL-1 antagonist is an IL-1 trap, wherein the IL-1 trap is a fusion protein comprising an IL-1 binding portion of the extracellular domain of IL-1RAcP, an IL-1 binding portion of the extracellular domain of IL-1R1, and a multimerizing component.

3. The method of either claim 1 or 2, wherein the subject is a human.

4. The method of claim 1, wherein the disease is selected from the group consisting of Alzheimer's disease (AD), Down's syndrome, multi-infarct dementia, cognitive impairment and cerebral amyloid angiopathy (CAA).

5. The method of claim 4, wherein the Alzheimer's disease is clinical, pre-clinical or prodromal Alzheimer's disease.

6. The method of claim 4, wherein the cerebral amyloid angiopathy is clinical or pre-clinical cerebral amyloid angiopathy.

7. The method of claim 1, wherein the IL-1 antagonist is an IL-1 trap comprising a fusion protein having an amino acid sequence selected from the group consisting of SEQ ID NOs: 2, 4, 6, 8, 10, 12, 14, 16, 18, 20, 22, 24, 26, and 28, or a substantially identical sequence having at least 95% identity to the sequence selected from the group consisting of SEQ ID NOs: 2, 4, 6, 8, 10, 12, 14, 16, 18, 20, 22, 24, 26, and 28 and capable of binding and inhibiting IL-1.

8. The method of claim 7, wherein the IL-1 trap is a fusion protein comprising the amino acid sequence of SEQ ID NO:10 or SEQ ID NO: 28.

9. The method of claim 1, wherein administration is subcutaneous, intramuscular, intranasal, intraarterial, intravenous, intrathecal, intraventricular, intracerebral, topical, transdermal administration or oral.

10. The method of claim 1, wherein a therapeutically effective amount is between about 1 mg/kg to about 750 mg/kg.

11. The method of claim 10, wherein a therapeutically effective amount is between about 10 mg/kg to about 500 mg/kg.

12. The method of claim 11, wherein a therapeutically effective amount is between about 50 mg/kg to about 150 mg/kg.

13. The method of claim 1, further comprising administering to a subject in need thereof a therapeutically effective amount of one or more other therapeutic agents, wherein the disease or at least one symptom associated with the disease is lessened in severity or duration, or wherein the onset or progression of the disease or at least one symptom associated with the disease is delayed.

14. The method of claim 1, wherein the at least one symptom associated with the disease is selected from the group consisting of memory loss, depression, anxiety, inattention, dementia, irritability, confusion, mood swings, and aggressive and/or apathetic behavior.

15. The method of claim 13, wherein administration of the other therapeutic agent is subcutaneous, intramuscular, intranasal, intraarterial, intravenous, intrathecal, intraventricular, intracerebral, topical, transdermal administration or oral.

16. The method of claim 13, wherein the other therapeutic agent is an acetylcholinesterase inhibitor or a glutamate pathway modifier.

17. The method of claim 16, wherein the acetylcholinesterase inhibitor is selected from the group consisting of ARICEPT® (donepezil HCl), EXELON® (rivastigmine tartrate), and RAZADYNE® (galantamine HBr).

18. The method of claim 16, wherein the glutamate pathway modifier is Namenda (memantine).

19. The method of claim 13, wherein the other therapeutic agent is selected from the group consisting of a different IL-1 antagonist, an anti-inflammatory agent, an antibody specific for tau, an antibody specific for beta amyloid and a microtubule stabilizer.

20. The method of claim 19, wherein the different IL-1 antagonist is selected from the group consisting of an IL-1 alpha or IL-1 beta antibody, a soluble IL-1 receptor, a different IL-1 trap, anakinra and canakinumab.

21. The method of claim 19, wherein the anti-inflammatory agent is aspirin or a different NSAID.

22. The method of claim 19, wherein the antibody specific for beta amyloid is selected from the group consisting of solanezumab, gantenerumab, and bapineuzumab.

23. The method of claim 19, wherein the microtubule stabilizer is epothilone.

24. A method of improving cognitive impairment in a mammal having beta amyloid deposits in brain tissue, the method comprising administering to the subject a therapeutically effective amount of an IL-1 antagonist as a first therapeutic agent, wherein the IL-1 antagonist is selected from the group consisting of an antibody specific for IL-1 alpha or IL-1 beta, or an antigen binding fragment thereof, a soluble IL-1 receptor, and an IL-1 fusion protein (IL-1 trap) comprising an IL-1 binding portion of the extracellular domain of IL-1RAcP, an IL-1 binding portion of the extracellular domain of IL-1R1, and a multimerizing component, wherein the mammal demonstrates an improvement in cognitive function(s) without necessarily exhibiting a concurrent change in the beta amyloid plaque burden in the brain.

25. A method of improving cognitive impairment in a mammal having beta amyloid deposits in brain tissue, the method comprising administering to the subject a therapeutically effective amount of an IL-1 antagonist as a first therapeutic agent, wherein the IL-1 antagonist is an IL-1 fusion protein (IL-1 trap) comprising an IL-1 binding portion of the extracellular domain of IL-1RAcP, an IL-1 binding portion of the extracellular domain of IL-1R1, and a multimerizing component, wherein the mammal demonstrates an improvement in cognitive function(s) without necessarily exhibiting a concurrent change in the beta amyloid plaque burden in the brain.

26. The method of claim 24, wherein the IL-1 antagonist is an IL-1 trap comprising a fusion protein having an amino acid sequence selected from the group consisting of SEQ ID NOs: 2, 4, 6, 8, 10, 12, 14, 16, 18, 20, 22, 24, 26, and 28, or a substantially identical sequence having at least 95% identity to the sequence selected from the group consisting of SEQ ID NOs: 2, 4, 6, 8, 10, 12,14, 16, 18, 20, 22, 24, 26, and 28 and capable of binding and inhibiting IL-1.

27. The method of claim 25, wherein the IL-1 trap is a fusion protein comprising the amino acid sequence of SEQ ID NO:10 or SEQ ID NO: 28.

28. The method of claim 24, further comprising administering to a subject in need thereof a therapeutically effective amount of one or more other therapeutic agents, wherein the disease or at least one symptom associated with the disease is lessened in severity or duration, or wherein the onset or progression of the disease or at least one symptom associated with the disease is delayed.

29. The method of claim 28, wherein the at least one symptom associated with the disease is selected from the group consisting of memory loss, depression, anxiety, dementia, inattention, irritability, confusion, mood swings, and aggressive and/or apathetic behavior.

30. The method of claim 28, wherein the administration of the other therapeutic agent is subcutaneous, intramuscular, intranasal, intraarterial, intravenous, intrathecal, intraventricular, intracerebral, topical, transdermal administration or oral.

31. The method of claim 28, wherein the other therapeutic agent is an acetylcholinesterase inhibitor or a glutamate pathway modifier.

32. The method of claim 31, wherein the acetylcholinesterase inhibitor is selected from the group consisting of ARICEPT® (donepezil HCl), EXELON® (rivastigmine tartrate), and RAZADYNE® (galantamine HBr).

33. The method of claim 31, wherein the glutamate pathway modifier is Namenda (memantine).

34. The method of claim 28, wherein the one or more other therapeutic agents are selected from the group consisting of a different IL-1 antagonist, an anti-inflammatory agent, an antibody specific for tau, an antibody specific for beta amyloid and a microtubule stabilizer.

35. The method of claim 34 wherein the different IL-1 antagonist is selected from the group consisting of an IL-1 alpha or IL-1 beta antibody, a soluble IL-1 receptor, a different IL-1 trap, anakinra and canakinumab.

36. The method of claim 34, wherein the anti-inflammatory agent is aspirin or a different NSAID.

37. The method of claim 34, wherein the antibody specific for beta amyloid is selected from the group consisting of solanezumab, gantenerumab, and bapineuzumab.

38. The method of claim 34, wherein the microtubule stabilizer is epothilone.
